(19) United States
(12) Patent Application Publication
(10) Pub. No.: US 2012/0070390 A1
(43) Pub. Date: Mar. 22, 2012

(54) Topical drug delivery system with dual carriers

(76) Inventors: D. Howard Phillips, Millerton, OK (US); Steven Keough, Sioux Falls, SD (US)

(21) Appl. No.: 13/066,705

(22) Filed: Apr. 21, 2011

Related U.S. Application Data
(60) Provisional application No. 61/455,888, filed on Oct. 28, 2010.
(60) Provisional application No. 61/403,712, filed on Sep. 20, 2010.
(60) Provisional application No. 61/342,954, filed on Apr. 21, 2010.

(51) Int. Cl. A61K 31/165; A61P 17/02; A61P 31/00; A61P 31/10; A61P 31/12; A61P 23/00; A61P 25/04; A61P 29/00; A61P 17/12; A61P 35/00; A61P 17/10; A61Q 19/08; A61P 17/18; A61K 8/97; A01P 7/02; A61P 37/08; A61Q 5/00; A61P 25/24; A61P 17/00; A61P 7/10; A61P 17/04; A61P 17/06; A61P 17/08; A61P 9/00; A01P 3/00; A61Q 7/00; A61K 38/22; A61P 37/06; A61P 37/02; A01P 7/04; A01P 17/00; A61P 33/14; A61Q 19/02; A61Q 19/04; A61K 31/59

(52) U.S. Cl. 424/59; 514/619; 514/167; 514/9.7; 514/5.2; 424/62

(57) ABSTRACT

An over-the-counter (OTC) regulated therapeutic composition for self-medication use for application to wounds having a potential pathogen load, in which the composition comprises a tissue penetration enhancer comprising a Class I pharmaceutical incipient; a pharmaceutical antibiotic agent in a dosing suitable for use in an OTC listing; a hygroscopic carrier agent comprising a Class I pharmaceutical incipient suitable for mixing in solution with the tissue penetration enhancer and the antibiotic agent; and wherein the activity/water (A_{w}) measurement of the composition is less than the A_{w} measurement for a target pathogen in a tissue wound.

### CROSS REFERENCE TO RELATED APPLICATIONS

This non-provisional application claims the benefit of the following co-pending provisional patent applications, the contents of which are fully included herein: 61/455,888 filed Oct. 28, 2010; 61/403,712 filed Sep. 20, 2010; 61/342,954 filed Apr. 21, 2010.

### FIELD OF THE INVENTION

A medical grade active pharmaceutical ingredient delivery formulation is provided to deliver active agents to designated tissue sites. The active agent delivery formulation is designed for topical application and uses dual carriers for transport and other functions.

### BACKGROUND OF THE INVENTION

Over the last half-century, the infection challenges to modern medicine have been dealt with by creation of new pharmaceutical compounds and new delivery modalities. Advances in drug delivery know-how have greatly aided the new compound discoveries. However, even as these efforts have improved overall health in many societies, new challenges have emerged—such as the newly spreading New Delhi metallobeta-lactamase (NDM-1) bacterial pathogen. Whether these challenges are in the form of new diseases or merely old diseases that have developed resistance mechanisms, the ability to combat these threats to society has often not been successful. This is in part why the World Health Organization has identified antibiotic resistance as the health issue of the new WHO annual focus.

The cost of new pharmaceuticals and delivery systems is high, both in financial resources as well as time. However, the need for new successful pharmaceutical-related outcomes is also quite high. This need is particularly acute within the infectious disease area. Particular problem areas involve community acquired and hospital/institution acquired bacterial infections. New levels of resistance to old medications further contribute to the need for new solutions. These trends are all occurring in the context of healthcare systems that are under greater demands by users, and often with considerable financial constraints. In many instances, chronic or recalcitrant infections are not being adequately treated, leading to co-morbidities and death. Public healthcare leaders decry the alarming levels of infection in many locations.

Although some progress is being made, what is really needed is a new approach to solving these vexing issues. What is needed is a simple active therapeutic system that is usable in a formulation that achieves a high rate of efficacy against the most prolific infections of the day.

### SUMMARY OF THE INVENTION

A drug delivery system, formed as a tissue penetrating solution, comprising:

a solvent suitable for solubilizing a non-liquid active ingredient into a solution; a diluent for diluting the solvent to optimize the solution for mammalian tissue compatibility; and a stabilizer for maintaining the solution chemically stable and substantially free from oxidation during storage for a pre-determined shelf life period.

A drug delivery system, formed as a tissue penetrating solution, comprising: a solvent suitable for solubilizing a non-liquid pharmaceutical ingredient into a solution, the solvent comprising a first tissue penetration enhancer; a diluent for diluting the solvent to optimize the solution for mammalian tissue compatibility, the diluent comprising a second tissue penetration enhancer; and a stabilizer for maintaining the solution chemically stable and substantially free from oxidation degradation during storage for a pre-determined shelf life period, the stabilizer comprising a dispersion enhancer for dispersing the pharmaceutical ingredient in the solution.

A tissue penetrating drug delivery system, formed as a solution, comprising: a tissue penetrating solvent suitable for solubilizing a non-liquid active pharmaceutical ingredient, the solvent comprising dimethyl sulfoxide in a concentration range of between about 5% and 20%; a tissue penetrating diluent for diluting the solvent to optimize the solution for mammalian tissue compatibility, the diluent comprising dipropylene glycol in a concentration range of between about 95% and 80%; and a stabilizer for maintaining the solution chemically intact and substantially free from oxidation during a pre-determined shelf life period, the stabilizer comprising ascorbic acid in a concentration range of between about 0.1% and 3%.

A controllable volume penetration drug delivery system, formed as a solution, and suitable for delivering at least one active pharmaceutical ingredient to desired volumes of mammalian tissue adjacent to the site of application of the drug delivery system, and a tissue regeneration system for improving the health of tissue adjacent to the site of application of the drug delivery system, comprising:

a solvent suitable for solubilizing an active pharmaceutical ingredient, the solvent comprising a first diffusion constant suitable for carrying the solubilized active pharmaceutical throughout a first tissue volume within mammalian tissue; a diluent for diluting the solvent and optimizing the solution for mammalian tissue compatibility, the diluent comprising a second diffusion constant suitable for carrying said active pharmaceutical ingredient throughout a second tissue volume within mammalian tissue; and the tissue regeneration system comprising an oxygen stabilizer in a total concentration range of between about 3% and 10%, and a vitamin D source in a medically efficacious amount.

A dual carrier controllable depth penetration drug delivery system, formed as a solution, suitable for delivering efficacious dosages of at least one active pharmaceutical ingredient to desired depths of mammalian tissue, comprising:

a. a first carrier suitable for solubilizing and carrying an active pharmaceutical ingredient through tissue, the first liquid carrier comprising a first diffusion constant suitable for carrying an efficacious concentration of an active pharmaceutical to a tissue depth deeper than the stratum corneum within a mammalian tissue site; and

b. a second carrier suitable for both diluting the solvent and optimizing the solution for mammalian tissue compatibility, the second liquid carrier having a second diffusion constant different than the first diffusion constant and suitable for carrying an efficacious concentration of said active pharmaceutical ingredient to a tissue depth shallower than the stratum corneum within the mammalian tissue site.

A topical drug delivery system, formed as a multi-functional solution, suitable for delivering at least one active pharmaceutical ingredient to desired locations of mammalian host tissue for primary therapeutic effect against pathogens at the desired locations, the drug delivery system also delivering secondary therapeutic effect by weakening the pathogen survival systems against the at least one active pharmaceutical ingredient thereby enhancing the primary effect of the active pharmaceutical ingredient and by improving healthy tissue natural response mechanisms in tissue adjacent to the pathogens, the drug delivery system comprising:

a. a first chemical penetration enhancer having solvent properties suitable for solubilizing an active pharmaceutical ingredient, the first chemical penetration enhancer comprising a first diffusion constant suitable for carrying the solubilized active pharmaceutical ingredient through mammalian skin and tissue to pathogen locations in that tissue to achieve primary therapeutic effect against the pathogens, and the first chemical penetration enhancer further having a characteristics suitable for carrying the active pharmaceutical ingredient through the cell walls of pathogens to deliver a portion of active pharmaceutical ingredient to an interior portion of the pathogen within the cell wall thereby enhancing the primary therapeutic effect of an active pharmaceutical ingredient against the pathogens;

b. a second chemical penetration enhancer having diluent properties for diluting the first chemical penetration enhancer and an active pharmaceutical in solution to optimize the solution for mammalian tissue compatibility and having further characteristics for providing a zone of enhanced inhibition to provide protection from any pathogenic effect between the adjacent healthy tissues and the pathogens; and

c. a dispersant mixable in solution with the first and second chemical penetration enhancers and an active pharmaceutical ingredient, said dispersant being suitable for providing secondary therapeutic effect by interaction with the active pharmaceutical ingredient to ensure substantial homogenous distribution of the selected active pharmaceutical ingredient in the solution during delivery of the solution to all areas of the mammalian tissue location.

A topical drug delivery system, formed as a multi-functional solution, suitable for delivering at least one active pharmaceutical ingredient to desired locations of mammalian host tissue for primary therapeutic effect against pathogens at the desired locations, the drug delivery system also delivering secondary therapeutic effect by weakening the pathogen survival systems against the at least one active pharmaceutical ingredient thereby enhancing the primary effect of the active pharmaceutical ingredient and by improving healthy tissue natural response mechanisms in tissue adjacent to the pathogens, the drug delivery system comprising:

a. a first chemical penetration enhancer having solvent properties suitable for solubilizing an active pharmaceutical ingredient, the first chemical penetration enhancer comprising a first diffusion constant suitable for carrying the solubilized active pharmaceutical ingredient through mammalian skin and tissue to pathogen locations in that tissue to achieve primary therapeutic effect against the pathogens, and the first chemical penetration enhancer further having a characteristics suitable for carrying the active pharmaceutical ingredient through the cell walls of pathogens to deliver a portion of active pharmaceutical ingredient to an interior portion of the pathogen within the cell wall thereby enhancing the primary therapeutic effect of an active pharmaceutical ingredient against the pathogens;

b. a second chemical penetration enhancer having diluent properties for diluting the first chemical penetration enhancer and an active pharmaceutical in solution to optimize the solution for mammalian tissue compatibility and having further characteristics for providing a zone of enhanced inhibition to provide protection from any pathogenic effect between the adjacent healthy tissues and the pathogens; and wherein the second chemical penetration enhancer and the first chemical penetration enhancer are in a ratio by weight percent of greater than 7:1; and

c. a dispersant mixable in solution with the first and second chemical penetration enhancers and an active pharmaceutical ingredient, said dispersant being suitable for providing secondary therapeutic effect by interaction with the active pharmaceutical ingredient to ensure substantial homogenous distribution of the selected active pharmaceutical ingredient in the solution during delivery of the solution to all areas of the mammalian tissue location; and wherein the dispersant also functions as a stabilizer for maintaining the solution chemically stable and substantially free from degradation during a pre-determined shelf life period; the dispersant in the therapeutic composition being in an amount from about 0.1% to about 10%, by weight of the drug delivery system solution.

A topical drug delivery system for use as a self-medication delivery system, formed as a multi-functional hygroscopic solution, suitable for delivering at least one active pharmaceutical ingredient to desired locations of mammalian host tissue for primary therapeutic effect against bacterial pathogens at the desired locations, the drug delivery system also delivering at least one secondary therapeutic effect by weakening the pathogen survival systems against the at least one active pharmaceutical ingredient thereby enhancing the primary effect of the active pharmaceutical ingredient and by improving healthy tissue natural response mechanisms in tissue adjacent to the pathogens, the drug delivery system comprising:

a. a non-hygroscopic first chemical penetration enhancer having solvent properties suitable for solubilizing an active pharmaceutical ingredient, the first chemical penetration enhancer comprising a first diffusion constant suitable for carrying the solubilized active pharmaceutical ingredient through mammalian skin and tissue to pathogen locations in that tissue to achieve primary therapeutic effect against the pathogens, and the first chemical penetration enhancer further having characteristics suitable for carrying the active pharmaceutical ingredient through the cell walls of pathogens to deliver a portion of active pharmaceutical ingredient to an interior portion of the pathogen within the cell wall thereby enhancing the primary therapeutic effect of an active pharmaceutical ingredient against the pathogens; the first chemical penetration enhancer having a weight percent range in the delivery system of between about 2% and 20%;

b. a hygroscopic second chemical penetration enhancer having diluent properties for diluting the first chemical penetration enhancer and an active pharmaceutical in solution to optimize the solution for mammalian tissue compatibility and having further characteristics for providing a zone of enhanced inhibition to provide protection from any pathogenic effect between the adjacent healthy tissues and the pathogens; the second chemical penetration enhancer having a weight percent range in the delivery system of between about 98% and 80%; and the second penetration enhancer having a second diffusion constant that is different than the diffusion constant of the first penetration enhancer; and

c. an anti-oxidizing dispersant mixable in solution with the first and second chemical penetration enhancers and an active pharmaceutical ingredient, said dispersant being in a weight percent of the solution of between 3% and 10% and being suitable for providing multiple secondary therapeutic effects by interaction with the active pharmaceutical ingredient to ensure substantial homogenous distribution of the selected active pharmaceutical ingredient in the solution during delivery of the solution to all areas of the mammalian tissue location and by further reducing the water activity level of the solution.

A topical drug delivery system, formed as a multi-functional solution, suitable for delivering at least one active pharmaceutical ingredient to desired locations of mammalian host tissue for primary therapeutic effect against pathogens at a primary tissue site, the drug delivery system also delivering secondary therapeutic effect by weakening the pathogen survival systems and rendering a pathogen more susceptible to the at least one active pharmaceutical ingredient, thereby enhancing the primary effect of the active pharmaceutical ingredient, and by improving host mammalian tissue natural response mechanisms in tissue adjacent to a pathogen load, the drug delivery system comprising:

a. a first chemical penetration enhancer having solvent properties suitable for solubilizing an active pharmaceutical ingredient, the first chemical penetration enhancer comprising a first diffusion constant suitable for carrying the solubilized active pharmaceutical ingredient through mammalian skin and tissue to pathogen locations in that tissue to achieve primary therapeutic effect against the pathogens, and the first chemical penetration enhancer further having a normal diffusion constant greater than about 1.5×10⁻⁵ cm²/sec for carrying the active pharmaceutical ingredient through the cell walls of pathogens to deliver a portion of active pharmaceutical ingredient to an interior portion of the pathogen within the cell wall thereby enhancing the primary therapeutic effect of an active pharmaceutical ingredient against the pathogens; and the first chemical penetration enhancer having a specific gravity greater than 1.0 so that it acts to alter the hydration sheath structure of proteins in the cell wall of the pathogen;

b. a hygroscopic second chemical penetration enhancer having diluent properties for diluting the first chemical penetration enhancer and an active pharmaceutical in solution to optimize the solution for mammalian tissue compatibility and having further hygroscopic characteristics for providing a tissue zone of enhanced inhibition against pathogen activity by reducing the water activity level in tissue adjacent to a primary pathogen site so that protection is created in the zone of enhanced inhibition from any pathogenic effect caused by pathogens in adjacent tissue; and wherein the second chemical penetration enhancer and the first chemical penetration enhancer are in a ratio by weight percent of greater than 7:1; and

c. an anti-oxidant dispersant mixable in solution with the first and second chemical penetration enhancers and an active pharmaceutical ingredient, said dispersant being suitable for providing secondary therapeutic effect by interaction with the active pharmaceutical ingredient to ensure substantial homogenous distribution of the selected active pharmaceutical ingredient in the solution during delivery of the solution to all areas of the mammalian tissue location; and wherein the dispersant also functions as a stabilizer for maintaining the solution chemically stable and substantially free from degradation during a pre-determined shelf life period; the dispersant in the therapeutic composition being in an amount from about 0.1% to about 10%, by weight of the drug delivery system solution, and wherein the solution is suitably hygroscopic to reduce the water activity level in any pathogen at a primary tissue site and at tissue adjacent to the primary tissue site to a level below a critical survival level of the pathogens below a value of about 0.9.

In a non-polymeric topical antibiotic drug delivery system, suitable for delivering at least one active antibiotic pharmaceutical ingredient to desired locations of mammalian host tissue for primary therapeutic effect against pathogens at a primary tissue site, and comprising at least one penetration enhancer having hygroscopic characteristics, the improvements comprising:

a. the delivery system having only three ingredients, with a first ingredient being a non-hygroscopic chemical penetration enhancer having solvent properties suitable for solubilizing an active pharmaceutical ingredient, the first chemical penetration enhancer comprising a first diffusion constant suitable for carrying the solubilized active pharmaceutical ingredient through mammalian skin and tissue to pathogen locations in that tissue to achieve primary therapeutic effect against the pathogens, and the first chemical penetration enhancer further having a normal diffusion constant greater than about 1.5×10⁻⁵ cm²/sec for carrying the active pharmaceutical ingredient through the cell walls of gram-positive and gram-negative bacterial pathogens to deliver a portion of active antibiotic pharmaceutical ingredient to an interior portion of the pathogen within the cell wall thereby enhancing the primary therapeutic effect of an active pharmaceutical ingredient against the pathogens; and the non-hygroscopic chemical penetration enhancer having a specific gravity greater than 1.05 so that it alters the hydration sheath structure of proteins in the cell wall of a bacterial pathogen; and

b. a second delivery system ingredient comprising the hygroscopic chemical penetration enhancer, but said enhancer also having diluent properties for diluting the non-hygroscopic chemical penetration enhancer and an active antibiotic pharmaceutical in solution to optimize the solution for mammalian tissue compatibility and having further secondary therapeutic effect using hygroscopic characteristics for providing a tissue zone of enhanced inhibition against pathogen activity by reducing the water activity level in tissue adjacent to a primary pathogen site so that protection is created in the zone of enhanced inhibition from any pathogenic effect caused by pathogens in adjacent tissue; and wherein the hygroscopic chemical penetration enhancer and the non-hygroscopic chemical penetration enhancer are in a ratio by weight percent of greater than 4:1; and

c. the system third ingredient comprising an anti-oxidant dispersant having a weak acidic pH mixable in solution with the chemical penetration enhancers and an active pharmaceutical ingredient, said dispersant being suitable for providing further secondary therapeutic effect by interaction with the active pharmaceutical ingredient to ensure substantial homogenous distribution of the selected active pharmaceutical ingredient in the solution during delivery of the solution to all areas of the mammalian tissue location; and wherein the dispersant also functions as a stabilizer for maintaining the solution chemically stable and substantially free from degradation during a pre-determined shelf life period; the dispersant in the therapeutic composition being in a weight percent amount from about 0.1% to about 10%, and wherein the solution is suitably hygroscopic to reduce the water activity level in any pathogen at a primary tissue site and at tissue adjacent to the primary tissue site to a level below a critical survival level of the pathogens below a value of about 0.9.

In a non-polymeric topical medicament comprising a therapeutic agent, a drug delivery system suitable for delivering the at least one therapeutic ingredient to desired locations of mammalian host tissue for primary therapeutic effect against pathogens at a primary tissue site, and comprising at least one penetration enhancer having hygroscopic characteristics, the improvements comprising:

a. the delivery system having only three ingredients, with a first ingredient being a non-hygroscopic chemical penetration enhancer having solvent properties suitable for solubilizing a therapeutic ingredient, the first chemical penetration enhancer comprising a first diffusion constant suitable for carrying the solubilized therapeutic ingredient through mammalian skin and tissue to pathogen locations in that tissue to achieve primary therapeutic effect against the pathogens, and the first chemical penetration enhancer further having a normal diffusion constant greater than about 1.5×10⁻⁵ cm²/sec for carrying the active pharmaceutical ingredient through the cell walls of pathogens to deliver therapeutic ingredient to an interior portion of the pathogen within the cell wall thereby enhancing the primary therapeutic effect of the therapeutic ingredient against the pathogens; and the non-hygroscopic chemical penetration enhancer having a specific gravity greater than 1.05 so that it alters the hydration sheath structure of proteins in the cell wall of a pathogen; and

b. a second delivery system ingredient comprising the hygroscopic chemical penetration enhancer, but said enhancer also having diluent properties for diluting the non-hygroscopic chemical penetration enhancer and a therapeutic ingredient in solution to optimize the solution for mammalian tissue compatibility and having further secondary therapeutic effect using hygroscopic characteristics for providing a tissue zone of enhanced inhibition against pathogen activity by reducing the water activity level in tissue adjacent to a primary pathogen site so that protection is created in the zone of enhanced inhibition from any pathogenic effect caused by pathogens in adjacent tissue; and wherein the hygroscopic chemical penetration enhancer and the non-hygroscopic chemical penetration enhancer are in a ratio by weight percent of greater than 4:1; and

c. the system third ingredient comprising an anti-oxidant dispersant having a weak acidic pH mixable in solution with the chemical penetration enhancers and an active pharmaceutical ingredient, said dispersant being suitable for providing further secondary therapeutic effect by interaction with the active pharmaceutical ingredient to ensure substantial homogenous distribution of the selected active pharmaceutical ingredient in the solution during delivery of the solution to all areas of the mammalian tissue location; and wherein the dispersant also functions as a stabilizer for maintaining the solution chemically stable and substantially free from degradation during a pre-determined shelf life period; the dispersant in the therapeutic composition being in a weight percent amount from about 0.1% to about 10%, and wherein the solution is suitably hygroscopic to reduce the water activity level in any pathogen at a primary tissue site and at tissue adjacent to the primary tissue site to a level below a critical survival level of the pathogens below a value of about 0.9.

An over-the-counter (OTC) therapeutic composition for self-medication use for application to wounds having a pathogen load, in which the composition comprises:

a. a tissue permeation enhancer comprising a Class I pharmaceutical excipient;

b. a pharmaceutical antibiotic agent suitable for use in an OTC monograph dose;

c. a hygroscopic carrier agent comprising a Class I pharmaceutical excipient suitable for mixing in solution with the tissue permeation enhancer and the antibiotic agent; and wherein the activity/water (A_{w}) measurement of the composition is less than the A_{w} measurement for a target pathogen in a tissue wound.

An antibiotic medication for mammalian use, the antibiotic medication comprising a tissue penetrating drug delivery system formed in a solution with a 3% concentration tetracycline active pharmaceutical ingredient and a tissue restoration system; the drug delivery system comprising a tissue penetrating solvent suitable for solubilizing a non-liquid active pharmaceutical ingredient, the solvent comprising dimethyl sulfoxide in a concentration range of between about 5% and 20%; a tissue penetrating diluent for diluting the solvent to optimize the solution for mammalian tissue compatibility, the diluent comprising dipropylene glycol in a concentration range of between about 95% and 80%; and a stabilizer for maintaining the solution chemically intact and substantially free from oxidation during a pre-determined shelf life period, the stabilizer comprising ascorbic acid in a concentration range of between about 0.1% and 3%; and the tissue restoration system comprising enhanced stabilizer volume to increase total stabilizer concentration to a range of between about 3% and 10%, and a vitamin D source in a medically efficacious amount.

A topical therapeutic medicament for use in a self-medication dosing form as a multi-functional solution, suitable for delivering at least one active pharmaceutical ingredient to desired locations of mammalian host tissue for primary therapeutic effect against bacterial pathogens at the desired locations, and also for delivering at least one secondary therapeutic effect by weakening the pathogen survival systems against the at least one active pharmaceutical ingredient thereby enhancing the primary effect of the active pharmaceutical ingredient and by improving healthy tissue natural response mechanisms in tissue adjacent to the pathogens, the medicament comprising:

a. a non-hygroscopic first chemical penetration enhancer having solvent properties suitable for solubilizing an active pharmaceutical ingredient, the first chemical penetration enhancer comprising a first diffusion constant suitable for carrying the solubilized active pharmaceutical ingredient through mammalian skin and other tissue to pathogen locations in that skin and tissue to achieve primary therapeutic effect against the pathogens, and the first chemical penetration enhancer further having characteristics suitable for carrying the active pharmaceutical ingredient through the cell walls of pathogens to deliver a portion of active pharmaceutical ingredient to an interior portion of the pathogen within the cell wall thereby enhancing the primary therapeutic effect of an active pharmaceutical ingredient against the pathogens; the first chemical penetration enhancer having a weight percent range in the medicament of between about 2% and 20%;

b. a hygroscopic second chemical penetration enhancer having diluent properties for diluting the first chemical penetration enhancer and an active pharmaceutical in solution to optimize the solution for mammalian tissue compatibility and having further characteristics for providing a zone of enhanced inhibition to provide protection from any pathogenic effect between the adjacent healthy tissues and the pathogens; the second chemical penetration enhancer having a weight percent range in the medicament of between about 98% and 80%; and the second penetration enhancer having a second diffusion constant that is different than the diffusion constant of the first penetration enhancer;

c. an anti-oxidizing dispersant mixable in solution with the first and second chemical penetration enhancers and an active pharmaceutical ingredient, said dispersant being in a weight percent of the medicament of between 3% and 10% and being suitable for providing multiple secondary therapeutic effects by interaction with the active pharmaceutical ingredient to ensure maintenance of substantial homogeneous distribution of the selected active pharmaceutical ingredient in the medicament during delivery to all areas of the desired mammalian tissue location and by further reducing the water activity level of the medicament to cause water stress of any pathogen contacted by the medicament; and

d. an active pharmaceutical ingredient present in the medicament in an amount from about 0.1% to about 5% by weight of the medicament.

In a non-polymeric topical medicament comprising a therapeutic agent, a drug delivery system suitable for delivering the at least one therapeutic ingredient to desired locations of mammalian host tissue for primary therapeutic effect against pathogens at a primary tissue site, and comprising at least one penetration enhancer having hygroscopic characteristics, the improvements comprising:

a. the delivery system having only three ingredients, with a first ingredient being a non-hygroscopic chemical penetration enhancer having solvent properties suitable for solubilizing a therapeutic ingredient, the first chemical penetration enhancer comprising a first diffusion constant suitable for carrying the solubilized therapeutic ingredient through mammalian skin and tissue to pathogen locations in that tissue to achieve primary therapeutic effect against the pathogens, and the first chemical penetration enhancer further having a normal diffusion constant suitable for carrying the active pharmaceutical ingredient through the cell walls of pathogens to deliver therapeutic ingredient to an interior portion of the pathogen within the cell wall thereby enhancing the primary therapeutic effect of the therapeutic ingredient against the pathogens; and the non-hygroscopic chemical penetration enhancer having a specific gravity greater than 1.05 so that it alters the hydration sheath structure of proteins in the cell wall of a pathogen; and

b. a second delivery system ingredient comprising the hygroscopic chemical penetration enhancer, but said enhancer also having diluent properties for diluting the non-hygroscopic chemical penetration enhancer and a therapeutic ingredient in solution to optimize the solution for mammalian tissue compatibility and having further secondary therapeutic effect using hygroscopic characteristics for providing a tissue zone of enhanced inhibition against pathogen activity by reducing the water activity level in tissue adjacent to a primary pathogen site so that protection is created in the zone of enhanced inhibition from any pathogenic effect caused by pathogens in adjacent tissue; and wherein the hygroscopic chemical penetration enhancer and the non-hygroscopic chemical penetration enhancer are in a ratio by weight percent of greater than 4:1; and

c. the system third ingredient comprising an anti-oxidant dispersant having a weak acidic pH mixable in solution with the chemical penetration enhancers and an active pharmaceutical ingredient, said dispersant being suitable for providing further secondary therapeutic effect by interaction with the active pharmaceutical ingredient to ensure substantial homogenous distribution of the selected active pharmaceutical ingredient in the solution during delivery of the solution to all areas of the mammalian tissue location; and wherein the dispersant also functions as a stabilizer for maintaining the solution chemically stable and substantially free from degradation during a pre-determined shelf life period; the dispersant in the therapeutic composition being in a weight percent amount from about 0.1% to about 10%, and wherein the solution is suitably hygroscopic to reduce the water activity level in any pathogen at a primary tissue site and at tissue adjacent to the primary tissue site to a level below a critical survival level of the pathogens below a value of about 0.9.

A topical therapeutic medicament for use in a self-medication dosing form as a multi-functional solution, suitable for delivering at least one active pharmaceutical ingredient to desired locations of mammalian host tissue for primary therapeutic effect against bacterial pathogens at the desired locations, and also for delivering at least one secondary therapeutic effect by weakening the pathogen survival systems against the at least one active pharmaceutical ingredient thereby enhancing the primary effect of the active pharmaceutical ingredient and by improving healthy tissue natural response mechanisms in tissue adjacent to the pathogens, the medicament comprising:

a. a non-hygroscopic first chemical penetration enhancer having solvent properties suitable for solubilizing an active pharmaceutical ingredient, the first chemical penetration enhancer comprising a first diffusion constant suitable for carrying the solubilized active pharmaceutical ingredient through mammalian skin and other tissue to pathogen locations in that skin and tissue to achieve primary therapeutic effect against the pathogens, and the first chemical penetration enhancer further having characteristics suitable for carrying the active pharmaceutical ingredient through the cell walls of pathogens to deliver a portion of active pharmaceutical ingredient to an interior portion of the pathogen within the cell wall thereby enhancing the primary therapeutic effect of an active pharmaceutical ingredient against the pathogens; the first chemical penetration enhancer having a weight percent range in the medicament of between about 2% and 20%;

b. a hygroscopic second chemical penetration enhancer having diluent properties for diluting the first chemical penetration enhancer and an active pharmaceutical in solution to optimize the solution for mammalian tissue compatibility and having further characteristics for providing a zone of enhanced inhibition to provide protection from any pathogenic effect between the adjacent healthy tissues and the pathogens; the second chemical penetration enhancer having a weight percent range in the medicament of between about 98% and 80%; and the second penetration enhancer having a second diffusion constant that is different than the diffusion constant of the first penetration enhancer;

c. an anti-oxidizing dispersant mixable in solution with the first and second chemical penetration enhancers and an active pharmaceutical ingredient, said dispersant being in a weight percent of the medicament of between 3% and 10% and being suitable for providing multiple secondary therapeutic effects by interaction with the active pharmaceutical ingredient to ensure maintenance of substantial homogeneous distribution of the selected active pharmaceutical ingredient in the medicament during delivery to all areas of the desired mammalian tissue location and by further reducing the water activity level of the medicament to cause water stress of any pathogen contacted by the medicament; and

d. an active pharmaceutical ingredient present in the medicament in an amount from about 0.1% to about 5% by weight of the medicament.

A surgical medicament for use as a penetrating medicated lavage in a deep tissue wound, formed as a multi-functional solution, suitable for delivering at least one active pharmaceutical ingredient to desired locations of mammalian host tissue for primary therapeutic effect against bacterial pathogens at the desired locations and adjacent surgically inaccessible locations, and also for delivering at least one secondary therapeutic effect by weakening the pathogen survival systems against the at least one active pharmaceutical ingredient thereby enhancing the primary effect of the active pharmaceutical ingredient and by improving healthy tissue natural response mechanisms in tissue adjacent to the pathogens, the medicament comprising:

a. a non-hygroscopic first chemical penetration enhancer having solvent properties suitable for solubilizing an active pharmaceutical ingredient, the first chemical penetration enhancer comprising a first diffusion constant suitable for carrying the solubilized active pharmaceutical ingredient through mammalian skin and other tissue to pathogen locations in that skin and tissue to achieve primary therapeutic effect against the pathogens, and the first chemical penetration enhancer further having characteristics suitable for carrying the active pharmaceutical ingredient through the cell walls of pathogens to deliver a portion of active pharmaceutical ingredient to an interior portion of the pathogen within the cell wall thereby enhancing the primary therapeutic effect of an active pharmaceutical ingredient against the pathogens; the first chemical penetration enhancer having a weight percent range in the medicament of between about 2% and 15%;

b. a hygroscopic second chemical penetration enhancer having diluent properties for diluting the first chemical penetration enhancer and an active pharmaceutical in solution to optimize the solution for mammalian tissue compatibility and having further characteristics for providing a zone of enhanced inhibition to provide protection from any pathogenic effect between the adjacent healthy tissues and the pathogens; the second chemical penetration enhancer having a weight percent range in the medicament of between about 98% and 85%; and the second penetration enhancer having a second diffusion constant that is different than the diffusion constant of the first penetration enhancer;

c. an anti-oxidizing dispersant mixable in solution with the first and second chemical penetration enhancers and an active pharmaceutical ingredient, said dispersant being in a weight percent of the medicament of between 3% and 10% and being suitable for providing multiple secondary therapeutic effects by interaction with the active pharmaceutical ingredient to ensure maintenance of substantial homogeneous distribution of the selected active pharmaceutical ingredient in the medicament during delivery to all areas of the desired mammalian tissue location and by further reducing the water activity level of the medicament to cause water stress of any pathogen contacted by the medicament and only temporary reversible water level reduction in adjacent host tissue; and

d. an active pharmaceutical ingredient present in the medicament in an amount from about 0.1% to about 5% by weight of the medicament.

A medical aid kit for treating a penetrating wound injury, comprising:

a. a first dispenser comprising a medical grade surfactant and disinfectant solution for applying to a contaminated surface having tissue toxic pathogens so that the pathogens are rendered substantially non-toxic and are removed from the contaminated surface; and

b. a second dispenser comprising a medical grade antibiotic medication for applying to the contaminated surfaces comprising a tissue penetrating drug delivery system formed in a solution with a 3% concentration tetracycline active pharmaceutical ingredient; the drug delivery system comprising a tissue penetrating solvent suitable for solubilizing a non-liquid active pharmaceutical ingredient, the solvent comprising dimethyl sulfoxide in a concentration range of between about 5% and 20%; a tissue penetrating diluent for diluting the solvent to optimize the solution for mammalian tissue compatibility, the diluent comprising dipropylene glycol in a concentration range of between about 95% and 80%; and a stabilizer for maintaining the solution chemically intact and substantially free from oxidation during a pre-determined shelf life period, the stabilizer comprising ascorbic acid in a concentration range of between about 0.1% and 3%, and a tissue regeneration system comprising additional stabilizer volume to increase total stabilizer concentration to a range of between about 3% and 10%, and a vitamin D source in a medically efficacious amount; wherein the antibiotic medication protects the wound injury from re-infection.

A method of selecting the constituent elements of a therapeutic composition for application to wounds having a pathogen load, in which the method comprises the steps of:

a. selecting a pharmaceutical active agent selected from a list of pharmaceutical active agents approved for over the counter non-prescription use, said pharmaceutical active agent having suitable activity to kill the desired pathogens;

b. identifying a tissue permeation enhancer from a list of generally recognized and safe tissue permeation enhancers that facilitates penetration of a composition using the selected tissue permeation enhancer through the stratum corneum of mammalian tissue;

c. selecting a hygroscopic carrier agent suitable for mixing in solution with the tissue permeation enhancer and the pharmaceutical active agent; said hygroscopic carrier agent being selected from a list of generally recognized and safe hygroscopic carrier agents; and

d. wherein the activity/water (A_{w}) measurement of the composition is less than the A_{w} measurement for a target pathogen in a tissue wound.

### BRIEF DESCRIPTION OF SEVERAL VIEWS OF THE DRAWINGS

The patent or application file contains at least one drawing executed in color. Copies of this patent or patent application publication with color drawings will be provided by the Office upon request and payment of the necessary fee.

FIG. 1 is a bottom plan view of a petri dish inoculated with MRSA and with an embodiment of the invention placed therein.

FIG. 2 is a top plan view of the petri dish of FIG. 1.

FIG. 3 is a bottom plan view of a petri dish inoculated with Proteus vulgaris and with an embodiment of the invention placed therein.

FIG. 4 is a top plan view of the petri dish of FIG. 3.

FIG. 5 is a bottom plan view of a petri dish inoculated with Pseudomonas aeruginosa and with an embodiment of the invention placed therein.

FIG. 6 is a top plan view of the petri dish of FIG. 5.

FIG. 7 is a bottom plan view of a petri dish inoculated with Enterobacter cloacae and with an embodiment of the invention placed therein.

FIG. 8 is a top plan view of the petri dish of FIG. 7.

FIG. 9 is a bottom plan view of a petri dish inoculated with Acinetobacter lwoffi and with an embodiment of the invention placed therein.

FIG. 10 is a top plan view of the petri dish of FIG. 9.

FIG. 11 is a bottom plan view of a petri dish inoculated with Acinetobacter baumanii and with an embodiment of the invention placed therein.

FIG. 12 is a top plan view of the petri dish of FIG. 11.

FIG. 13 is a bottom plan view of a petri dish inoculated with Group-A Streptococcus and with an embodiment of the invention placed therein.

FIG. 14 is a top plan view of the petri dish of FIG. 13.

FIG. 15 is a graph of the ratio of kill zone area to application zone area relating to FIGS. 1-14.

FIG. 16 is a plan view of a petri dish inoculated with Staphylococcus aureus and with three embodiments of the invention placed therein and with control dosing of other active agents.

FIG. 17 is a plan view of a petri dish inoculated with MRSA and with three embodiments of the invention placed therein and with control dosing of other active agents.

FIG. 18 is a plan view of a petri dish inoculated with Klebsiella pneumoniae and with three embodiments of the invention placed therein and with control dosing of another active agent.

FIG. 19 is a plan view of a petri dish inoculated with E. coli and with three embodiments of the invention placed therein and with control dosing of another active agent.

FIG. 20 is a plan view of a petri dish inoculated with Proteus vulgaris and with three embodiments of the invention placed therein and with control dosing of another active agent.

FIG. 21 is a plan view of a petri dish inoculated with Pseudomonas aeruginosa and with three embodiments of the invention placed therein and with control dosing of another active agent.

FIG. 22 is a plan view of a petri dish inoculated with Enterobacter cloacae and with three embodiments of the invention placed therein and with control dosing of another active agent.

FIG. 23 is a plan view of a petri dish inoculated with Acinetobacter lwoffi and with three embodiments of the invention placed therein and with control dosing of another active agent.

FIG. 24 is a plan view of a petri dish inoculated with Acinetobacter baumanii and with three embodiments of the invention placed therein and with control dosing of another active agent.

FIG. 25 is a plan view of a petri dish inoculated with Enterococcus faecalis and with three embodiments of the invention placed therein and with control dosing of another active agent.

FIG. 26 is a front plan view of a petri dish inoculated with Streptococcus pyogenes and with three embodiments of the invention placed therein and with control dosing of another active agent.

FIG. 27 is a back plan view of the petri dish of FIG. 26, also showing a presumptive test agent for Strep-A.

FIG. 28 is a summary compilation of the zones of inhibition for the data shown in FIGS. 16-27.

FIG. 29 is a graph of the Lot 00228 comparison of the zones of inhibition areas to the zones of application areas.

FIG. 30 is a reference legend for FIG. 29.

FIG. 31 is a graph of the Lot 00228 ratios of the zones of inhibition to the zones of application.

FIG. 32 is a reference legend for FIG. 31.

FIG. 33 is a graph of the Lot 00229 comparison of the zones of inhibition areas to the zones of application areas.

FIG. 34 is a reference legend for FIG. 33.

FIG. 35 is a graph of the Lot 00229 ratios of the zones of inhibition to the zones of application.

FIG. 36 is a reference legend for FIG. 35.

FIG. 37 is a schematic view depicting the location or a stoma deep tissue post-surgical incision infected with Staphylococcus aureus.

FIG. 38 is a side elevation view of the infected incision of FIG. 37.

FIG. 39 is a side elevation view of the infected incision of FIG. 37.

FIG. 40 is a side elevation view of the infected incision of FIG. 37 after initial treatment with an embodiment of the invention.

FIG. 41 is a side elevation view of the infected incision of FIG. 37 after further treatment with an embodiment of the invention.

FIG. 42 is a side elevation view of the previously infected incision of FIG. 37 after eight days of treatment with an embodiment of the invention.

FIG. 43 is a side elevation view of a weeping MRSA infected lesion from the left earlobe of a patient.

FIG. 44 is a closer view of the image of FIG. 43.

FIG. 45 is a side elevation view of the previously infected ear lobe lesion of FIG. 43 after four days of treatment with an embodiment of the invention.

FIG. 46 is a top view of an infected swollen left first digit of a diabetic patient's foot.

FIG. 47 is a top perspective view of the swollen digit of FIG. 46.

FIG. 48 is a medial side elevation view of the swollen digit of FIG. 46, further showing extensive tissue breakdown on the medial side of the digit.

FIG. 49 is the same view of FIG. 47, but after five days of treatment with an embodiment of the invention.

FIG. 50 is the same view of FIG. 48, but after five days of treatment with an embodiment of the invention.

FIG. 51 is the same view of FIG. 47, but after seventeen days of treatment with an embodiment of the invention.

FIG. 52 is the same view of FIG. 48, but after seventeen days of treatment with an embodiment of the invention, and showing substantially all tissue intact and all indications of infection resolved, along with loss of inflammation.

FIG. 53 is a top plan view of a petri dish inoculated with a fungal infection and with multiple embodiments of the invention placed therein and with control dosing of other active agents.

FIG. 54 is a front elevation view of a diabetic patient's left foot showing tissue breakdown on multiple digits.

FIG. 55 is a bottom view of the right foot of the patient of FIG. 53 showing a deep tissue infected diabetic lesion.

FIG. 56 is another bottom view of the lesion of FIG. 54.

FIG. 57 is another bottom view of the lesion of FIG. 54.

FIG. 58 is close-up view of the lesion of FIGS. 54-57.

FIG. 59 is a bottom view of the healing lesion of FIGS. 54-58, after eleven days of treatment with an embodiment of the invention.

FIG. 60 is a close-up view of the lesion of FIG. 54 before treatment with the invention.

FIG. 61 is the view of the lesion of FIG. 60 after eleven days of treatment with an embodiment of the invention.

FIG. 62 is a bottom view of the right foot of a diabetic patient showing a deep tissue infected diabetic lesion.

FIG. 63 is a close-up view of the lesion of FIG. 62.

FIG. 64 is a bottom view of the lesion of FIG. 62 after treatment for seven days with an embodiment of the invention.

FIG. 65 is a close-up view of the lesion of FIG. 64 after treatment.

FIG. 66 is a top view of a brown recluse spider bite on the middle phalanx region of a finger prior to a five day treatment with an embodiment of the invention.

FIG. 67 is the view of the bite location shown in FIG. 66 after five weeks.

FIG. 68 is a left perspective view of a patient's face with acne prior to treatment with an embodiment of the invention.

FIG. 69 is a left side view of the patient's face of FIG. 68.

FIG. 70 is a close-up left side view of the patient's face of FIGS. 68-69 after three weeks of treatment with an embodiment of the invention.

### DETAILED DESCRIPTION OF THE INVENTION

### Design Parameters and Multi-Functionality of Selected Constituent Ingredients

In view of the need for improved approaches to combating infectious diseases, and the need to defeat the cycle of drug resistance, this invention describes further specifics to the actual need and provides high-efficacy solutions. Accordingly, what is further needed is a broad spectrum pharmaceutical composition that enjoys a high rate of tolerance among large populations, a low resistance rate, a high rate of efficacy, and which is available at a reasonably affordable cost compared with other possible active agents and delivery systems and in the context of the cost to the healthcare systems of failed solutions of the past.

What is further needed is a pharmaceutical carrier or drug delivery system that is well tolerated, is readily and rapidly approvable under multi-national regulatory schemes, and is able to combine with and effectively deliver a wide range of pharmaceutical and other therapeutic agents to target tissue sites. Such a pharmaceutical carrier or drug delivery system must be simple, with a minimum of ingredients and with maximum efficacy, and preferably may be suitable for use with either novel or well-known therapeutic agents. The pharmaceutical carrier or drug delivery system should be efficacious, affordable to many people, highly tolerable, and chemically stable for the intended purpose. Such a carrier and active agent combination must present high potency kill mechanisms to targeted pathogens while simultaneously presenting as benign or beneficial to host tissue in the target region and adjacent healthy tissue.

What is particularly needed is a pharmaceutical carrier or drug delivery system and active agent combination which employs new combinations of pathogen kill mechanisms simultaneous with new mechanisms to prevent the development of resistance by the pathogen of interest. In one embodiment, this novel combination is provided by an active agent kill and an inactive agent effecting the mechanisms to subvert resistance buildup. In another embodiment, more than one active agent may be employed to achieve pathogen kill and to aid in effecting the mechanisms to subvert resistance buildup. In yet another embodiment, this novel combination is provided by at least one active agent kill (i.e. a primary therapeutic effect) and two or more inactive agents affecting the mechanisms to subvert resistance buildup (i.e. second or third therapeutic effects). In yet a further embodiment, this novel combination is provided by both an active agent kill mechanism and an inactive agent kill mechanism, along with at least one inactive agent effecting the mechanisms to subvert resistance buildup and assisting the active agent kill by at least one of the following mechanisms: serving as a carrier of the active agent, serving as a homogeneous dispersant of the active agent, serving as a chemical signal for up-regulation of natural wound healing cascades at the local delivery site, serving as a disrupter of membrane energy generation by the pathogen, serving as a displacer of hydration sheaths of pathogen proteins, serving as a delivery vehicle for secondary therapeutic effect agents and tissue healing ingredients, serving as an emollient, serving as a displacer of water molecules in the pathogen, and serving as a hygroscopic agent to lower the water activity level of a pathogen. These capabilities in an efficiently designed pharmaceutical product comprise remarkable breakthroughs in the way diseases are combated.

What is further needed is a pharmaceutical carrier or drug delivery system and active agent combination which does not cause: undesirable side effects to users, such as unacceptable drying or alteration of healthy tissue; painful sensations; degradation byproducts that are not readily or safely cleared; increased risk of side effects created by multiple active pharmaceutical agents within the product; digestive or other biological system disturbance; altered breath or thought processes; or likely risk of building drug resistance in targeted pathogens—even if the article is not taken on strict or frequent intervals. These and other desirable characteristics are missing from the prevalent medications of our day.

Of particular significance to the present invention is the existing widespread variety of tenacious pathogens that cause significant societal cost. Examples of these pathogens include those known to cause disease states referred to as Methicillin Resistant Stapylococcus Aureus (MRSA), Staphylococcus aureus (Staph), Acinetobacter baumannii, Acinetobacter lwoffi, Klebsiella pneumoniae, E. coli, Proteus vulgaris, Pseudomonas aeruginosa, Enterobacter cloacae, Group-A streptoccocus, and others either discussed herein or widely known. These bacterial pathogens are generally referred to as either gram positive or gram negative. Few classes of pharmaceutical active agents provide adequate broad spectrum killing power to be clinically effective against both gram positive and gram negative organisms, and certainly not against those colonized into vibrant, persistent and resistant infections. Many other injury states are of relevance to this invention, including those that have localized tissue necrosis as well as systemic killing effects, such as those occurring from snake or spider bites, as well as other forms of injury leading to secondary infections. A further disease state of interest relates to tissue abnormalities caused by chronic disease states, such as diabetes or other conditions that cause inadequate blood distribution and low levels of perfusion at limb peripheries and other locations which are then fertile ground for infected lesions to develop. Further chronic or periodic tissue anomalies may include a range of dermatological states, including without limitation eczema, acne, psoriasis, and others of both dermatologic or immune system origins. Methodologies and articles of the invention should be functional against multiple disease states, either as a prophylactic or as active therapy against lesions or other tissue injuries and abnormalities. It is also recognized that the articles and methods of these inventions have efficacy against viral and fungal infections, as well as providing analgesic relief to patients as will be further discussed herein.

The mechanisms or modes of action to kill the above pathogens normally vary considerably based on the pathogen-pharmaceutical combination. Yet a goal of this invention was to create a simpler and more unified approach to effective minimum inhibitory concentrations of active agent against these pathogens. What was discovered was that effective use of known ingredients could result in a powerful broad spectrum effect using an old drug in a newly configured delivery formulation. The constituent ingredients were carefully selected as those with multi-functional attributes that are, nevertheless, well tolerated by large cohorts of potential patient populations.

The resulting invention was achieved by careful assessment of existing delivery systems in the pharmaceutical and cosmetic industries, and by detailed review of the mechanisms of resistance in the various pathogens of interest. The technology and useful article design premises included: minimizing the number of ingredients; using ingredients that preferably had multiple therapeutic uses within the formulation (i.e. “multi-functional”); using well known and well tolerated individual ingredients that could qualify for rapid introduction to patient care; achieving both active kill of pathogens and active healing of host tissue affected by the pathogens; accelerating the healing processes wherever possible and safe to do so; using kill mechanism(s) that would be difficult for any pathogen to rapidly evolve a resistance mechanism to defeat the kill mechanism; using technologies that could accommodate a wide range of active pharmaceutical or other therapeutic agents; creating a zone of inhibition to further prevent disease spread to tissue adjacent to infected tissue, and using a relatively low cost ingredient list to enable widespread adoption of use.

Using the above criteria, it was recognized that both chemical as well as mechanical techniques could be best employed as tools to overcome and prevent pathogen resistance. An analysis of the food science arts of preservatives and food microbiology led to recognition that lowering the water activity level (A_{w}) of the pathogens below a viability and/or survival level could amply shock the pathogens mechanically (i.e. “water stress”), and would present the most difficult evolutionary barrier for the resistance mechanisms to overcome. The availability of water for the growth and metabolic processes is a very useful and unifying concept to consider in the design of kill mechanisms for the pathogens of interest. While microbial water stress can be achieved by an excess of water, the more practical approach was to limit the water activity of the pathogens—while not harming the proximal host tissue. Accordingly, a hygroscopic approach is a cornerstone of certain embodiments of the delivery system and medicaments herein disclosed.

Although the concept of water activity levels of certain common pathogens is known in the life science area, for example as shown in U.S. patent publication 2008/0292560A1, and various scientific literature, what has not been appreciated is how to combine that knowledge with the other design parameters noted herein above to create simple, elegant and effective therapeutic articles and methodologies. Moreover, what appears to be further lacking in any design or structure of active agent formulations is integration of the design parameters disclosed herein with recognition of the nature of the nutrient mechanism of the prokaryotes that must derive their nutrients from surrounding solution. For example, as that solution becomes less aqueous and more laden with agents toxic to the pathogen, the combined effect of the “nutrient” intake and insufficient water activity has a first synergistic killing effect. Accordingly, achieving solute concentrations which are intolerable to these pathogens is an excellent initial attack on these microorganisms, and is one method of enabling the reclamation of extraordinary levels of efficacy for long-retired classes of, or specific, pharmaceuticals.

That said, it is also recognized that many bacteria, let alone yeasts, molds and fungi, can survive in a somewhat dehydrated state while retaining the ability to revitalize in conditions of later water activity at a higher level. One example of this is the sulphur reducing bacteria found deep in the earth which proliferate wildly in the presence of shale and natural gas water fracturing operations.

It is known that most of the bacteria of interest noted above have baseline A_{w} levels between about 0.9 and 1.0, and that the A_{w} level of Staphylococcus aureus is at about 0.85 to 0.88. See e.g., Brown, Bacteriological Reviews, December 1976, p. 803-846. These A_{w} levels are made at the normal mammalian range of body temperatures. It is acknowledged that differing growth rates of Staphylococcus aureus at higher temperatures and differing water activity levels are known in the food science field, such as discussed in Czech Journal of Food Science, Vol. 27, 2009, Special Issue 2:S2-28-S2-35. Of more interest, however, may be that even though cell death of pathogens occurs, eventually, if the water activity is too low for too long a period, there is evidence of increased cell death when a low A_{w} stressed pathogen experiences increased water activity levels back toward its norm. This is shown, for example, in work by Mugnier et al, Applied and Environmental Microbiology, July 1985, p. 108-114. In fact, the rate of survival of pathogens in water-stressed low A_{w} environments is directly influenced by the compounds in the medium around them. In one set of experiments it was demonstrated that the stability of the compounds in low A_{w} environments was enhanced by a medium with high molecular weight, such as a polysaccharide, mannitol, and other compounds. In other words, it is discovered that low molecular weight molecules markedly endanger survival of gram-positive and gram-negative molecules during a low A_{w} stress event. This little known discovery is useful in the design and selection of ingredients in the present invention—which ingredients intentionally have low molecular weights to further ensure against a pathogen rebound during normal tissue re-hydration. Additionally, for compounds that have essentially the equivalent number of carbon atoms, those with an acid group (such as ascorbate) are much more reactive toward these pathogens than ones without such groups.

It is known that low molecular weight sugar has some wound healing characteristics, for example as shown by Ambrose et al, Antimicrobial Agents and Chemotherapy, September 1991, p. 1799-1803. This work demonstrated in part that pastes of basic sucrose or xylose applied to wounds actually lowered the A_{w} levels in proximal tissue. While the value of using sugar in wounds is debatable, particularly for large classes of patients, the point of lowering the A_{w} levels at wound sites to inhibit pathogen growth, with a relatively low molecular weight medium, has merit.

These insight are useful to the design of the technologies in this invention. For example, this knowledge aids in the selection of carrier system constituents, particularly as to structural and steric attributes. This know-how also suggests benefits in reducing the number of constituents to avoid providing nutritive compounds to the pathogens under water stress, which would undermine the efforts toward inhibition of the pathogens. This has been no recognition by others of this feature of embodiments of the present invention. A further lesson that is not appreciated in the art is to avoid formulations and constituents that rely on or tend to add water to the site of action of these formulations, particularly after an initial shock to the pathogen. Absent this proscription, the aqueous solution surrounding the cells is sufficiently dilute to remove the conditions that are intended at the outset to retard growth or rebound of the colony forming pathogens.

What has been needed, therefore, is a low molecular weight delivery system for active pharmaceutical and other therapeutic agents that also has demonstrable hygroscopic characteristics or other secondary disease killing and disease prevention mechanisms. The system can not utilize constituents with any history of byproduct clearance problems or allergies, such as with some polyethylene glycol vehicles or other polymer systems, for example as shown in Wilson et al, Pharm. Int. 5:94-97. A further desirable feature is to use at least one delivery constituent which has superior permeation enhancement attributes for carrying therapeutic agents through levels of tissue without significant loss or creation of non-homogeneity of the agents across the delivery site tissue volume. Yet a further attribute in such vehicle is to select at least one constituent ingredient or excipient with a density sufficient to displace water in hydration sheaths of pathogen proteins. This will potentially disrupt pathogenic cell replication mechanisms while also enable certain up-regulation of cellular signaling at host tissue sites that is beneficial to triggering the healing mechanisms of the host.

For example, in one embodiment, at least one of the penetration enhancers in the drug delivery system of the invention up-regulates the action of the host mammalian tissue immune response adjacent to any pathogen. This may be accomplished by selection of a penetration enhancer which effectively pre-treats the target mammalian tissue host cells to prime cell surfaces to increase natural expression of intregrin adhesion molecules. In yet another embodiment, these integrin adhesion molecules may function as triggers of the polymorphonuclear complex, or simply the neutrophil recruitment to the site of pathogens. In this regard, the penetration enhancer or other constituent ingredient should demonstrate some characteristic to dispose expression of integrin molecules selected from the list of retinoic acid-dependent expression molecules comprising integrin 11b, 11c, and 18. In one experiment, DMSO has demonstrated an attribute of upregulation of mRNA and protein expression in epigenetic profiles in mouse embryonic stem cells and embryoid bodies. This is described at Iwatani et al, Stem Cells 2006; 24:2549-2556. However, more specific examples of priming of cells may be found in experiments described in Balint et al, Molecular and Cellular Biology, July, 2005, p. 5648-5663, in which is described a short exposure to dimethyl sulfoxide or vitamin D induces a precommitment in HL-60 cells. This pre-commitment caused by DMSO exposure results in the acquisition of a precommitment memory that can be sustained for more than one cell cycle. Of particular interest to the present invention, is that priming of the cells by pretreatment with differentiating agents such as vitamin D or the solvent DMSO increases subsequent retinoid-induced TGM2 expression. In the primed cells, even after being washed out and then treated with 9-cis retinoic acid, the cell surface expression of integrin molecules CD18 or integrin 132 and its heterodimeric partners CD11b and CD11c showed an increased expression as compared to naïve cells. Of further value to the present invention is that the priming effect is transient and only lasts for about 24 to 48 hours. In the context of the invention herein, this is excellent timing for therapeutic effect of the drug delivery system and medicaments, but it also allows restoration of the natural immune response within a short time following treatment.

In these experiments it is further recognized that only a very small amount of DMSO is needed to achieve desired cellular responses. For example, results occurred at less than 2% DMSO and at about 100 nM of vitamin D. Use of these findings in the context of design of a drug delivery system is contrary to the teachings generally in the art. It is generally recognized that high levels of DMSO or similar penetration enhancers may be advisable to deliver a medically effective dose of active agent(s) to a pathogenic tissue area, particularly if the delivery is through to deeper tissue or passage through the epidermis is required. However, the inventors have recognized that various attributes and secondary therapeutic effects of each constituent element of a new drug delivery system may enable unconventional proportions of such ingredients, with unexpected results. This is one example, in which only 1-2% of a polar non-hygroscopic solvent is required to achieve high efficacy drug delivery through tissue and drug-resistant cell wall structures. However, the design of the overall delivery system must have other elements that aid this ingredient with mechanisms other than merely diffusion. Further examples are to select ingredients that alter the membrane potential of pathogen cell membranes, thereby disrupting the electrical signals controlling pumps and motion of the cell. In this regard, choosing an ingredient that includes functions such as activation of ion channels of the cell wall of the pathogen enabling entry of the solution into the nucleoid or cytoplasm of the pathogen cell is favorable. Other attributes of a multi-functional ingredient, such as a penetration enhancer, may include: activation of ligand-gated channels of the cell wall of the pathogen enabling entry of the solution into the nucleoid of the pathogen cell, desensitization of AMDA receptors of ligand-gated channels of the cell wall of the pathogen, activation of voltage-gated channels of the cell wall of the pathogen enabling entry of the solution into the nucleoid of the pathogen cell, desensitization of NMDA receptors of voltage-gated channels of the cell wall of the pathogen, disruption of ATP generation, or mechanisms to overcome efflux pumping mechanisms within the pathogen cell structures. These secondary functions of excipients in the drug delivery system enable use of the fewest possible constituents while achieving the maximum therapeutic effect according to the intentional design parameters of the invention.

While these features allow maximum design flexibility of the formulation, another key selection criteria is to ensure that constituents are generally recognized as safe and effective for human use as an inactive or active ingredients in regulated pharmaceutical products. In other words, selection of ingredients that are already designated by national or international regulatory or standard-setting entities as GRAS allows for greater utility and acceptance of the resulting inventive combinations. As noted earlier, yet another critical feature of the deliver system constituents is to have excellent permeation attributes at both the epidermal corneum stratum level of tissue as well as at the cell walls of pathogens—but be controllable or tunable to ensure systemic safety is maintained and toxicity is avoided. This has been a likely inhibitor and barrier to introduction of use of “super penetration/permeation” delivery systems, i.e. failure to be multi-functional and relying on only the diffusion mechanism to achieve efficacy. In other words, in the invention herein, preferred constituents would need to achieve all the above favorable outcomes and still be able to efficiently penetrate the cell wall of all target pathogens, including those of the challenging gram-negative bateria, and effectively deliver active agents to the nucloid and cytoplasm regions at cytotoxic levels. Given these complicated design parameters, many of which have not been considered by others as a requirement of a single constituent for these purposes, the selection of the polar aprotic solvent dimethyl sulphoxide in very small quantities and the hygroscopic dipropylene glycol in much larger quantities were selected as one of the preferred embodiments of a dual carrier controllable delivery system. At least one additional essential ingredient is the proper dispersant or stabilizer for any active agent to be safely and effectively delivered by this system. Remarkably, with the carefully determined amount of ascorbic acid, these three ingredients enable renewed efficacy of one of the oldest and most well tolerated antibiotics worldwide, tetracycline (i.e. tetracycline hydrochloride). Indeed, the restored efficacy is so exceptional that the resulting composition or medicament is useful in self-medication dosing levels within over-the-counter listings or monographs (also referred to as general sales lists or non-prescription forms). While certain embodiments have preferred ingredients, it is recognized that analogs, derivatives, and other similarly multi-functional ingredients may be suitable under the design parameters discussed herein. Therefore, the dispersant, anti-oxidant or stabilizer may be selected from the list including ascorbic acid, sorbic acid, a thiol, lipoic acid, a polyphenol, glutathione, tocopherol (vitamin E), a tocotrienal, uric acid, a peroxidase, coenzyme Q, carotene, and meltonin. At least one penetration enhancer or first ingredient may be selected from the list including sulfoxides, polyols, urea, sugars, lactams, amides, fatty acids, fatty alcohols, terpenes, anionic-surfactants, cationic-surfactants, non-ionic surfactants, and Zwitterionic-surfactants. The first penetration enhancer or first ingredient may thus be selected from the list of sulfoxide dispersants including dimethyl sulfoxide and dodecyl methyl sulfoxide. A second penetration enhancer or second ingredient may be selected from the list of polyol chemical penetration enhancers including propylene glycol, dipropylene glycol, polypropylene glycol, 1,2-propanediol, and polyethylene glycol.

### Drug Delivery

Numerous modalities of drug delivery are known, including for example oral, topical, parenteral, intra-vascular, buccal and other sites of transmucosal, and transdermal. This invention is designed for use as a topical drug delivery formulation but may also have other forms of use in open wound beds, which may not normally be considered as “topical” applications. Additionally, “topical” in this teaching may include external topical as well as internal topical, as may be appropriate. Generally, however, reference to “topical” will refer to external topical and surgical open access wound beds.

The field of delivery of medicaments is vast, with numerous pharmaceutical carriers having diverse characteristics and efficacies. The invention provides novel pharmaceutical formulations as well as pharmaceutical carrier technologies that provide new uses for old pharmaceutical active agents. The invention thus includes new and unforeseeable improvements to old pharmaceuticals, therapeutic agents and delivery systems that may yield or restore high efficacy levels against both gram positive and gram negative pathogens, and other non-microbial pathogens. The drug delivery system may also accommodate novel or well-known recent generations of active pharmaceutical agents. As will be shown, this enables patients to receive the benefits of the original efficacy of old pharmaceuticals as if no resistance mechanisms had evolved. This effectively re-sets the clock of resistance back in time spanning numerous decades. The invention provides relatively low cost and well-tolerated methodology and formulations to slow the societal damage incurred by ever-increasing burdens on populations due to many types of chronic disease states. It is expected that use of these technologies will favorably and materially alter the institutional bacteriograms at hospitals and other community healthcare sites which track local drug resistance patterns.

Pharmaceutical carriers have different advantages according to the desired dosing form. Tablet and capsule forms of carriers typically provide nominal protection for the active agent during storage and ingestion. However, following intake by the patient there is considerable loss of active agent due to the tissue barriers the agent must cross to enter the blood stream. This loss is often referred to as “first pass” loss. Concentrations are also diminished as the active agent is dispersed throughout the patient's body. Buffers and controlled release structures and chemistries enable more optimum timed release of active agents, but such agents must still cross numerous boundaries to achieve affect at target tissue sites. The addition of these additives may also further complicate the clearance mechanisms of degradation byproducts, or cause other undesired patient reactions.

Direct parenteral or intra-vascular dosing reduces the loss due to the ingestion processes, and is a preferred delivery form for a wide variety of pharmaceutical active agents. However, this delivery form also has drawbacks in its lack of site-specific delivery to targeted tissue areas. To overcome this deficiency, topical applications, subcutaneous injections or even transdermal drug delivery is often used. Ideally, a site specific dose should optimize the time and dose of active therapy at the specific area/volume of tissue designated as the target site.

Unfortunately, transdermal delivery is normally designed for the goal of systemic delivery via the bloodstream—so that modality remains somewhat limited in its site-specific concentration effect. Also, injections are least favored by patients and are less accurate relating to depth of delivery against pathogens. Even topical delivery of pharmaceutical active agents is significantly limited in its efficacy against many forms of infectious disease pathogens due to its general targeting of shallow or epidermal tissue alone. In this regard, the depth within the patient's skin where the pathogen resides may present significant problems for a mere topical agent delivery system—particularly those with long-chain active molecules or carriers. In particular, the skin forms an effective barrier at the level of the corneum stratum that prevents absorption of many medications. However, if a disease process is at all resident beneath such barrier layer then many pharmaceutical active agents that are topically delivered will not provide the fully desired therapeutic effect. This is particularly important when attempting to kill many common bacteria with ineffective antibiotic delivery systems. One common example of this phenomenon is when a wound has penetrated the corneum stratum allowing pathogen entry more deeply into tissue wound sites. Recognition of these problems, and providing effective and creative solutions, are parts of the invention herein, although the solutions are not limited to such wound types only as noted above.

Efforts have been made in the art to chemically modify the barrier properties of skin to permit the penetration of certain agents (since topical diffusion rate is primarily controlled or limited by the stratum corneum), enhance the effectiveness of the agent being delivered, enhance delivery times, reduce the dosages delivered, reduce the side effects from various delivery methods, reduce patient reactions, and so forth. Use of heat, sonic waves and other external devices have also been employed to promote transport of agents through tissue, and are recognized as possible adjuvant therapeutic delivery modalities for use with the inventions described herein.

Tissue penetration or permeation enhancers have been used to increase the permeability of the dermal surface to drugs, and are often proton accepting solvents such as dimethyl sulfoxide (DMSO) and dimethylacetamide. Other examples of less favorable penetration enhancers that have been studied and reported as effective include 2-pyrrolidine, N,N-diethyl-m-toluamide (Deet), 1-dodecal-azacycloheptane-2-one N,N-dimethylformamide, N-methyl-2-pyrrolidine, calcium thioglycolate, hexanol, fatty acids and esters, pyrrolidone derivatives, derivatives of 1,3-dioxanes and 1,3-dioxolanes, 1-N-dodecyl-2-pyrrolidone-5-carboxylic acid, 2-pentyl-2-oxo-pyrrolidineacetic acid, 2-dodecyl-2-oxo-1-pyrrolidineacetic acid, 1-azacycloheptan-2-one-2-dodecylacetic acid, and aminoalcohol derivatives, including derivatives of 1,3-dioxanes, among others. A few of the many excellent reviews of common tissue penetration/permeation enhancers include the works of Kanikkannan et al, Current Medicinal Chemistry 2000 June; 7(6):593-608, and Karande et al, Journal of Controlled Release 115 (2006) 85-93.

The most common penetration enhancers, however, are sometimes either toxic to some people, irritating, oily, odiferous, or allergenic. Specifically, the penetration enhancers used and thought to be necessary to transdermally deliver active agents such as steroid hormones, namely, compounds such as long chain fatty acids such as oleic acids, fatty alcohols such as lauryl alcohol and long-chain fatty esters such as isopropyl myristate, tend to include aliphatic groups that make the formulations oily and malodorous. Numerous other examples exist in the art.

U.S. Pat. No. 5,891,462 teaches the use of lauryl alcohol as a permeation enhancer for estradiol and norethindrone acetate. Such formulations are not appealing to the user nor to anyone else in close proximity to the user. Although that particular patent discloses three examples of estradiol or norethindrone acetate formulations having no lauryl alcohol component, such formulations are comparative examples that are intended to illustrate the long held position that long chain fatty alcohols such as lauryl alcohol are necessary to transdermally deliver norethindrone acetate in combination with estradiol to a subject.

Additionally, for example, the known testosterone gel formulations FORTIGEL® and TOSTRELLE® (Cellegy Pharma, South San Francisco, Calif.), both include ethanol, propanol, propylene glycol, carbomer, triethanolamine, purified water, and oleic acid as a permeation enhancer, the latter being responsible for the irritating and malodorous characteristics of these formulations. Also, TESTIM® (Auxilium Pharmaceuticals, Norristown, Pa.) is a 1% testosterone gel and includes pentadecalactone, acrylates, glycerin, polyethylene glycol (PEG), and pentadecalactone as a permeation enhancer. It is a very odoriferous compound. Also, TESTIM® is not desirable because it contains undesirable amounts of glycerin which are not well tolerated by the skin.

Thus, there is a need for a topical formulation that adequately delivers active agents to patients with skin tolerability in mind, but does not include the unpleasant odor common to the prior art formulations and other drawbacks common to transdermal mechanisms. Other permeation enhancers are used in the cosmetics industry. These are typically designed for shallow tissue penetration. For example, the monoalkyl ether of diethylene glycol is diethylene glycol monomethyl ether or diethylene glycol monoethyl ether or mixtures thereof. Polyalcohols may also be used in conjunction with permeation enhancers in order to retain moisture in the skin, as in U.S. Pat. No. 4,575,515. In some instances and teachings, the polyalcohol and the permeation enhancer may be present in various ratios depending on need, such as for example weight ratios of about 2:1 to 1:1. Alternatively, the polyalcohol and permeation enhancer may be present in a weight ratio of about 1.25:1 to 1.2 to 1.

For the purpose of illustration and not limitation, the alkanol may be a C2 to C4 alcohol such as ethanol, isopropanol, or n-propanol. Examples of this are found in U.S. Pat. No. 3,671,654. As known in the art, the amount of the alcohol component of the formulation may be selected to maximize the diffusion of the active agent through the skin while minimizing any negative impact on the active agent itself or desirable properties of the formulation. A goal of the present invention is to obviate the need for any alcohol based permeation enhancer.

Although transdermal patches and delivery systems are known, such are designed more as controlled release technologies rather than penetration enhancing technologies. This is best exemplified by anti-nicotine and medical narcotic administering systems. Trans-dermal systems are also designed for delivery of the active agent into the bloodstream to achieve systemic dosing. As previously noted, this is different than topical dosing, both in the delivery mechanism and the delivery goal.

The delivery system embodiments of this invention include various potential ingredients and design approaches. Accordingly, a non-hygroscopic first chemical penetration enhancer having solvent properties suitable for solubilizing an active pharmaceutical ingredient is desired. The first chemical penetration enhancer may have a first diffusion constant suitable for carrying the solubilized active pharmaceutical ingredient through mammalian skin and tissue to pathogen locations in that tissue to achieve primary therapeutic effect against the pathogens. Also, the first chemical penetration enhancer should have further characteristics suitable for carrying the active pharmaceutical ingredient through the cell walls of pathogens to deliver a portion of active pharmaceutical ingredient to an interior portion of the pathogen within the cell wall thereby enhancing the primary therapeutic effect of an active pharmaceutical ingredient against a variety of pathogens. As noted, the first chemical penetration enhancer may have a weight percent range in the delivery system of between about 2% and about 20%. A hygroscopic second chemical penetration enhancer may be combined with the first chemical penetration enhancer. An additional feature of a penetration enhancer is to have a specific gravity greater than 1.05 so that it alters the hydration sheath structure of proteins in the cell wall of a bacterial pathogen.

The second penetration enhancer should have diluent properties for diluting the first chemical penetration enhancer and an active pharmaceutical in solution to optimize the solution for mammalian tissue compatibility. Yet it should have further characteristics for providing a zone of enhanced inhibition to provide protection from any pathogenic effect between the adjacent healthy tissues and the pathogens. This second chemical penetration enhancer should have a weight percent range in the delivery system of between about 98% and 80%. The second penetration enhancer may also have a second diffusion constant that is different than the diffusion constant of the first penetration enhancer. In one embodiment, a desired further feature of the delivery systems is where the hygroscopic chemical penetration enhancer and the non-hygroscopic chemical penetration enhancer are in a ratio by weight percent of greater than 4:1.

An anti-oxidizing dispersant mixable in solution with the first and second chemical penetration enhancers and an active pharmaceutical ingredient is also desired. The dispersant should be in a weight percent of the solution of between 3% and 10% and be suitable for providing multiple secondary therapeutic effects. These are achievable by the dispersant through interaction with the active pharmaceutical ingredient to achieve substantial homogeneous distribution of the selected active pharmaceutical ingredient in the solution during delivery of the solution to all areas of the mammalian tissue location. Another attribute of one embodiment of the dispersant is to further reduce the water activity level of the solution. Yet another embodiment of the delivery system is to configure the dispersant in the therapeutic composition at about 0.1% to about 10% and to ensure that the solution is suitably hygroscopic to reduce the water activity level in any pathogen at a primary tissue site and at tissue adjacent to the primary tissue site to a level below a critical survival level of the pathogens below a value of about 0.9. A more preferred level of water activity is at a level below about 0.85. In yet another embodiment, the dispersant may be selected as a weak acid having a pH greater than about 4.0.

### Active Pharmaceutical Agents

In view of the wide variety and evolution of active pharmaceutical agents, and concomitant wide variety in their modes of action against different pathogens, it is not obvious to revert to use of an unaltered old drug. However, despite the contrary teachings and trends in the art, the technologies of this invention enable such use of old and unaltered active pharmaceutical ingredients in a powerful new way. Accordingly, although the technologies may be employed with different classes of active agents, a first embodiment of active agent for use in this invention includes tetracycline. Indeed, as will be shown, the only significant limitation on the type of active agent suitable for use with these technologies is molecular weight.

As such, active agents that can be delivered through tissue by the preferred carrier systems disclosed herein are included by reference in this teaching. Suitable active agents may be selected or screened from the group consisting of antimicrobials, antifungals, antivirals, anesthesics, analgesics, corticosteroids, non-steroidal anti-inflammatories, retinoids, lubricating agents, anti-warts, anti-proliferative, vasoactive, keratolytic, dicarboxylic acids and esters; calcium channel blockers, cholinergic, N-oxide donors, photodynamic, anti-acne, anti-wrinkle, anti-oxidants, self-tanning active herbal extracts, acaricides, age spot and keratose removing agents, allergens, anti-aging agents, antibiotics, anti-burn agents, anti-cancer agents, anti-dandruff agents, anti-depressants, anti-dermatitis agents, anti-edemics, antihistamines, antihelminths, anti-hyperkeratolyte agents, anti-inflammatory agents, anti-irritants, anti-lipemics, antimycotics, anti-proliferative agents, anti-anti-pruritics, anti-psoriatic agents, anti-rosacea agents, anti-seborrheic agents, antiseptics, anti-swelling agents, anti-yeast agents, astringents, topical cardiovascular agents, chemotherapeutic agents, dicarboxylic acids, disinfectants, fungicides, hair growth regulators, hormones, hydroxy acids, immuno-suppressants, immuno-regulating agents, insecticides, insect repellents, keratolytic agents, lactams, metals, metal oxides, mitocides, neuropeptides, oxidizing agents, pediculicides, photodynamic therapy agents, retinoids, sanatives, scabicides, self-tanning agents, skin whitening agents, vasoconstrictors, vasodilators, vitamins, vitamin D derivatives, wound healing agents and wart removers. The active agent may also be selected from the group consisting of acyclovir, azelaic acid, benzoyl peroxide, betamethasone, caffeine, calcipotriol, calcipotriol hydrate, calcitriol, ciclopiroxolamine, diclofenac sodium, ketoconazole, miconazole nitrate, minoxidil, mupirocin, nifedipine regular, permethrin bpc (cis:trans 25:75), piroxicam, salicylic acid and terbinafine hcl. Alternatively, the active agent may be selected from the group of simply consisting of a beta-lactam antibiotic, an aminoglycoside, an anthraquinone, an azole, an antibiotic glycopeptide, a macrolide, an antibiotic nucleoside, an antibiotic peptide, an antibiotic polyene, an antibiotic polyether, an antibiotic quinolone, an antibiotic steroid, a sulfonamide, an antibiotic metal, an oxidizing agent, a periodate, a hypochlorite, a permanganate, a substance that releases free radicals and/or active oxygen, colloidal oatmeal, a cationic antimicrobial agent, a quaternary ammonium compound, a biguanide, a triguanide, a bisbiguanide, a polymeric biguanide, and analogs, derivatives, salts, ions and complexes thereof.

Additionally, it is recognized that the teachings of this invention may further enable the use of altered basic structures of existing drugs, including those of tetracycline and its derivatives and analogs, and other unique or legacy drugs. A few examples, without limitation to the many others, of altered or modified pharmaceuticals or others which may benefit from the present technologies may be found in the U.S. Pat. Nos. 4,871,767, 6,346,391, or 7,825,136, the teachings of which are all incorporated by reference as potential active agents for use with at least one embodiment of the inventions herein.

In the midst of current efforts to create new super-pharmaceuticals to deal with newly evolving super-pathogens, a new approach is possible. Remarkably, using the teachings of this invention, the original early generation tetracycline may now have newly identified efficacies. Indeed, when deployed using the improved delivery formulations as described herein, tetracycline is again potent against organisms which have established resistance to the drug in other delivery modalities since introduction over sixty years ago. In view of the unique modes of action of the delivery systems and the various active agents, it is believed that similar restoration of efficacy of many other early generation active agents is now possible.

Commercially developed from the chlortetracycline work in the late 1940's and with production techniques patented in the 1950s, as for example those taught in U.S. Pat. No. 2,516,080, resistance mechanisms to the tetracycline class of protein synthesis inhibitors evolved to the point that subsequent embodiments and derivatives were required to maintain efficacy. Various alternate forms and derivatives exist, some of which include, e.g., chlortetracycline, oxytetracycline, minocycline, doxycycline, methacycline, lymecycline and others. Tetracycline and derivative drugs are naturally occurring or semi-synthetic polyketide compounds that exhibit a well-known broad-spectrum antibacterial activity that interferes with prokaryotic protein synthesis at the ribosome level. In addition to this well-known antibacterial activity these compounds also exhibit a variety of additional, less well-known properties. Among them are separate and distinct anti-inflammatory properties. Tetracycline and related compounds have been shown to be effective chemotherapeutic agents in a wide variety of chronic inflammatory diseases and conditions. The newest addition to the class is a glycylcycline known commercially under the name of Tigecycline.

In addition to being well-tolerated and an excellent first aid antibiotic worldwide for many years, tetracycline and related compounds has also demonstrated efficacy against periodontitis, rosacea, acne, auto-immune diseases such as rheumatoid arthritis and protection of the central nervous system against trauma and neurodegenerative diseases such as stroke, multiple sclerosis and Parkinsons disease. Tetracycline and related compounds appear to be beneficial for treatment of several chronic inflammatory airway diseases. Among them are asthma, bronchiectasis, acute respiratory distress syndrome, chemical induced lung damage, cystic fibrosis and chronic airway inflammation.

Normally, tetracycline dosing has been limited to a tablet or capsule form (both solids) due to oxidation susceptibility in a liquid or ointment form. In a liquid form, the drug is naturally yellow but turns black with oxidation. The liquid form has therefore been less preferred due to shelf-life concerns, refrigeration recommendations and consumer preference. Overall, these limitations have rendered liquid or ointment forms of tetracycline as disfavored. Unfortunately, the incentives to discover the potential advantages of a liquid or ointment form of tetracycline delivery, including as a topically delivered agent, were lost due to these circumstances—until the present invention. This invention includes new and unexpected anti-oxidation, stabilization, and homogeneous dispersion techniques for use with liquid and ointment forms of tetracycline and other agents vulnerable to oxidation degradation and solution consistency. Indeed, despite consumer disfavor when tetracycline eventually does lose its natural yellow color, the invention has further resulted in increased preservation of medical efficacy despite color change, which will be further discussed herein. In one embodiment, the anti-oxidant and stabilization techniques used demonstrate the multi-functionality of the essential constituent ingredients in the invention. In this instance, the levels of anti-oxidant agents in the invention may result in secondary benefits relating to promoting tissue repair and regeneration at the interface of a pathogen and proximal healthy tissue, as well as contributing to one of the various modes of action of pathogen inhibition. Additional criticalities and co-dependencies are disclosed herein relating to stability of various embodiments and percent of other core ingredients in the basic inventive formulations.

Prior use of tetracycline (referred to generally herein as “TCN”) has been generally limited to a tablet, capsule or pill form of dosing. This is primarily due to the previously discussed susceptibility to damaging oxidation processes. However, the widespread patient tolerance to tetracycline provides an ideal potential to create a new use and a new dosing formulation for this excellent active agent, substantially in its original form, as well as for its derivative forms and embodiments. Moreover, basic tetracycline and chlortetracycline are frequently listed by national governments as suitable active pharmaceutical agents for use in over-the-counter medications, assuming suitable delivery systems are available—which they have not been until now. This OTC aspect is very important to the overall design goals of this invention in order to enable widespread adoption in a self-medication dose, maintain affordability, and to re-enable a well accepted broad-spectrum pharmaceutical worldwide.

In one embodiment of the invention, a formulation is provided by which tetracycline may be placed into an optimum viscosity ointment solution which has excellent stability and shelf life. This has not been reliably accomplished in the past. Also, in this invention, the term “optimum viscosity” is intended to mean an ointment that is configured for rapid penetration into tissue to achieve maximum simultaneous primary and secondary therapeutic effects, including barrier and emollient functions at a micro-scale, into all sizes of tissue/wound sites and tissue/cellular interstices. Other embodiments provide formulations in which the tetracycline is within more viscous and/or semi-solid forms, i.e. a more thick barrier-style ointment form. These options result in new forms of dosing availability that leads to further advantages that were previously unattainable with this pharmaceutical agent. This enables improved patient care by expanding the range of treatment options that are available and providing an economical therapy regime with higher levels of efficacy and lower risk profiles than any known alternatives. The ease of topical application in certain embodiments further adds to the acceptance by the patient, resulting in improvements throughout healthcare systems worldwide. Of further significant economic and public health interest is the availability of existing over-the-counter monographs or other regulatory mechanisms for use of liquid tetracycline for various common indications. However, to date, no product has successfully met that challenge and opportunity until now.

Techniques of active agent preservation are known in the art. Various preservatives and anti-oxidants are well known. Anti-oxidants are generally included in formulations as substances which inhibit oxidation or suppress reactions promoted by oxygen or peroxides. One example is taught in U.S. Pat. No. 5,874,479 in which a wide assortment of anti-oxidant candidates, especially lipid-soluble antioxidants, are taught. However, in this case the teaching is to promote absorption into the cellular membrane of non-pathogenic tissue to neutralize oxygen radicals and to protect the tissue. Of note, one of the antioxidants included is ascorbic acid, in different forms. However, the teaching suggests away from use of ascorbic acid as being toxic to (healthy) monocytes unless accompanied by sodium pyruvate. In the present invention, the use of critical amounts of ascorbic acid or sorbic acid, along with critical concentrations of other constituents, enables exceptional stability for the otherwise oxidation-vulnerable tetracycline active agent, while overcoming the cumbersome drawbacks noted in the above referenced patent. These critical and co-dependent concentrations and weight percents of ingredients in this invention also enables remarkably controllable distribution of the active pharmaceutical agent, as will be shown in examples herein below. Accordingly, there is an antioxidant/dispersant/stabilizer agent that has multiple functions as a preservative, an oxygen scavenger, a stabilizer of the color center in tetracycline, and an active agent dispersant control ingredient with demonstrable fidelity.

In another embodiment, the invention provides a liquid tetracycline (or certain other active agents) formulation by which a stable and highly efficacious active agent delivery is achieved. This high efficacy is against persistent pathogens, which are often not susceptible to effective eradication by other techniques. This new and unexpected result occurs due to the discovery of a sequence of formulation steps and constituent ingredients related to the formulation development. In particular, these steps include: a) providing a selected concentration of a tetracycline suitable for use with mammalian patients; b) combining the tetracycline with a select solvent to provide a tetracycline solution; c) combining a diluent or buffer to the solution to optimize the solution for tissue compatibility; and d) combining an anti-oxidant with the solution to minimize damage from oxidation effects on the tetracycline and to ensure precise and controlled dispersion in the solution.

### Pre-Clinical Experiments

### ZOI:ZOA

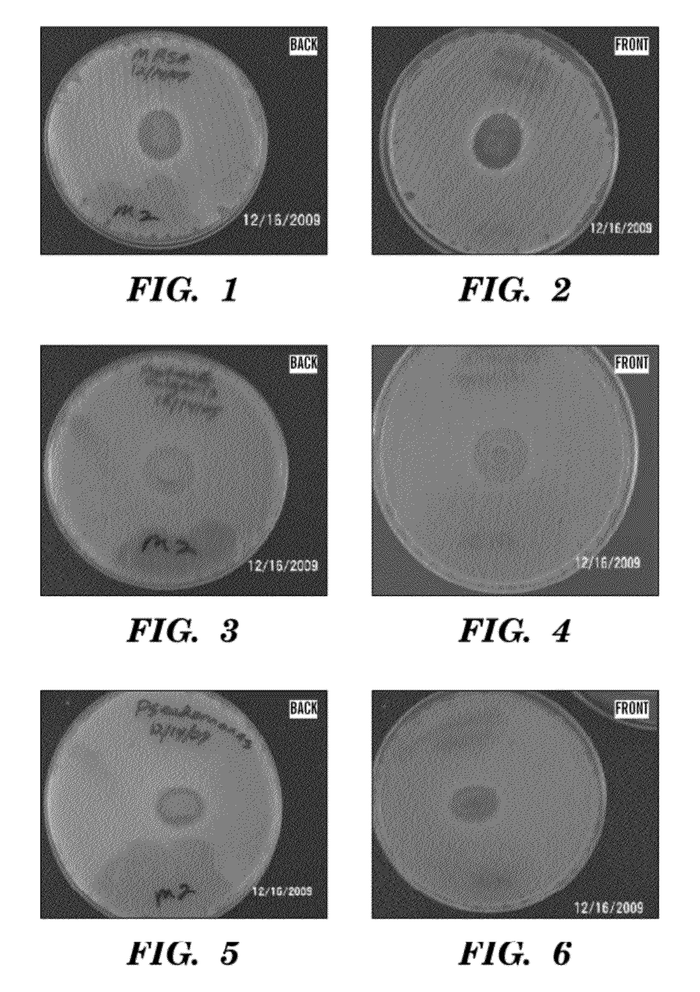

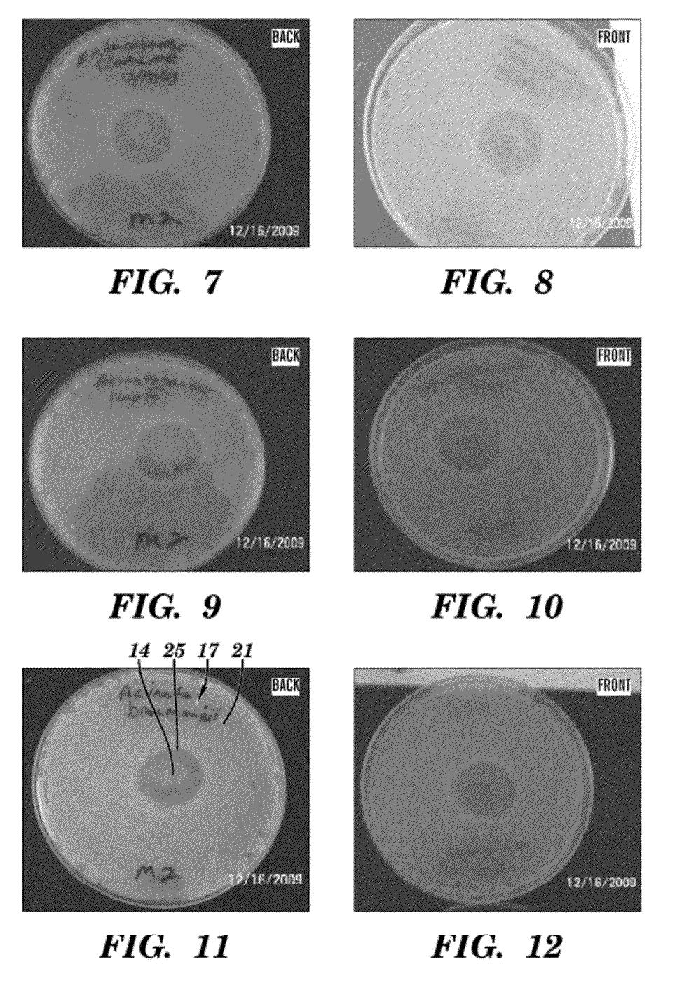

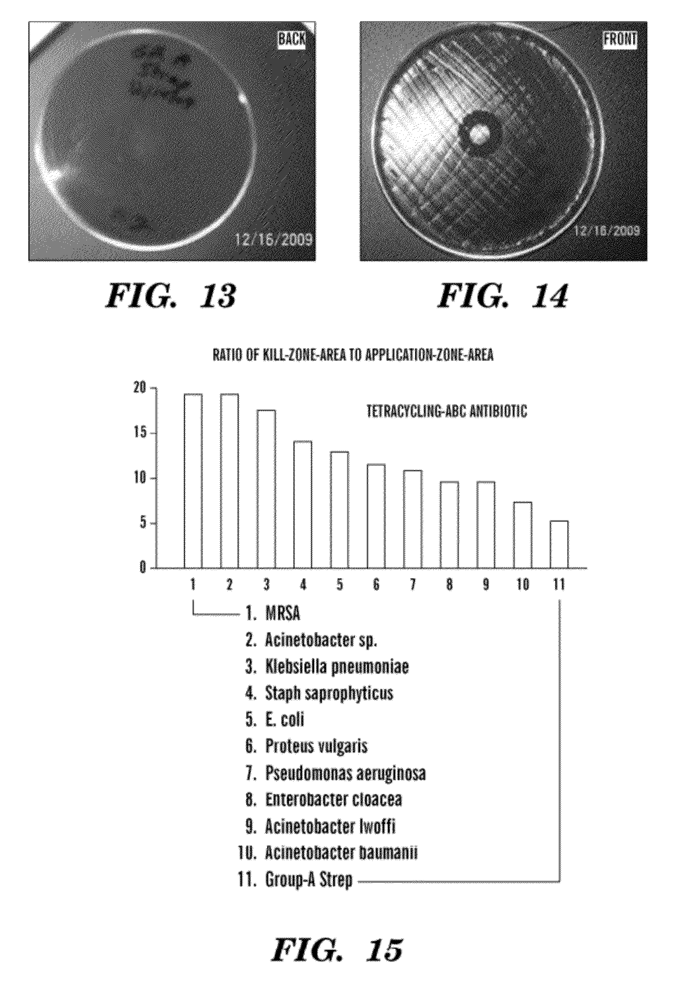

In vitro Petri dish testing of embodiments of the invention was conducted by inoculation of the dish with a healthy growth of live bacteria before the antibiotic medication and drug delivery formulation of a first embodiment of the invention was applied. As shown in FIG. 1, a yellow circular shape 14 indicates where one drop of the antibiotic was dropped in the Petri dish 17, onto the bacteria laden gel material 21. The shape 14 comprises the Zone of Application (“ZOA”). A grey circular shape 25 is formed comprising the extent of diffusion of the effective medication. This is referred to as the Zone of Inhibition (“ZOI”). The grey circular shape 25 comprises the region where the bacteria were killed. The Zone of Inhibition is always larger than the Zone of Application of the antibiotic. The Zone Of Application (ZOA) diameter 27 is typically 10 millimeters. Results of these tests are below, with some reference to Figures showing back and front views against various leading pathogens of concern:

| Bacteria | Antibiotic | | | |
|---|---|---|---|---|
| (% by weight) | concentration | ZOA | ZOI | ZOI:ZOA |
| MRSA | 3% | 10 mm | 31-44 | 19 |
| (FIGS. 1-2) | | | | |
| Acinetobacter sp. | 3% | 10 mm | 44 | 19 |
| Klebsiella pneumoniae | 3% | 10 mm | 34 | 12 |
| Staph Saprophyticus | 3% | 10 mm | 44 | 19 |
| E. coli | 3% | 10 mm | 42 | 18 |
| Proteus vulgaris | 3% | 10 mm | 29 | 8 |
| (FIGS. 3-4) | | | | |
| Pseudomonas aeruginosa | 3% | 10 mm | 23 | 5 |
| (FIGS. 5-6) | | | | |
| Enterobacter cloacae | 3% | 10 mm | 31 | 10 |
| (FIGS. 7-8) | | | | |
| Acinetobacter lwoffi | 3% | 10 mm | 36 | 13 |
| (FIGS. 9-10) | | | | |
| Acinetobacter baumannii | 3% | 10 mm | 33 | 11 |
| (FIGS. 11-12) | | | | |
| Group-A strep | 3% | 10 mm | 27 | 7 |
| (FIGS. 13-14) | | | | |

The exceptional ZOI versus ZOA ratios are demonstrated above, as well as graphically in FIG. 15, normalized. As will be shown in subsequent data, the degree to which the area of inhibition is a multiple of the area of application, it also appears that the volume of inhibition in vivo is similarly much greater than would be expected from normal topical antibiotic therapy using drug delivery mechanisms currently available.

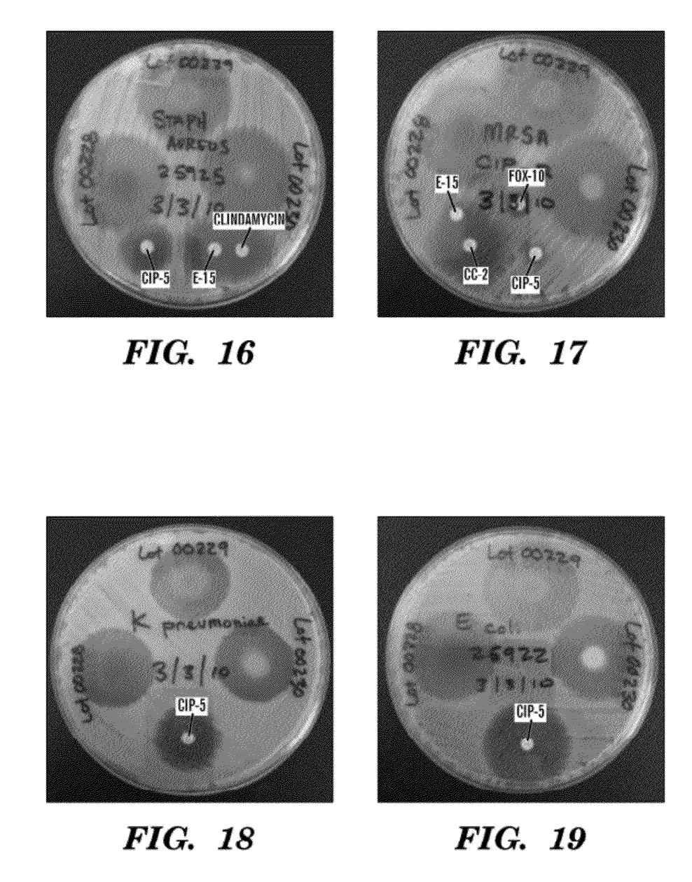

The following data comprises in-vitro comparisons of different formulations. In each of the views in FIGS. 16-27 there is shown several formulations or embodiments, referred to as “Lots”, having three different amounts of anti-oxidant to determine preferred formulations to achieve both primary and secondary therapeutic effects according to the invention.

Lot 00228 used a double carrier (i.e. the dual carrier drug delivery system) and 365 times the chemical stabilizer needed to reduce oxygen radicals to 50% of the value with no chemical stabilizer. Lot 00229 used a double carrier and 73 times the chemical stabilizer needed to reduce oxygen radicals to 50% of the value with no chemical stabilizer. Lot 00230 was provided as a high-contrast zone-of-application (sharply-defined TCN center circles) to verify that the zone of application for each vertical drop was approximately 10 mm in diameter.

FIG. 18 demonstrates the use of the dual carrier drug delivery system having identical 3% tetracycline with ascorbic acid as the stabilizer, but with different stabilizer values or amounts, and the effect on dispersing the active agent within the delivery system. Lot 00228 shows a more uniform or homogeneous distribution of the active agent than the intermediate Lot 00229 and Lot 00230—that has no stabilizer in its formulation.

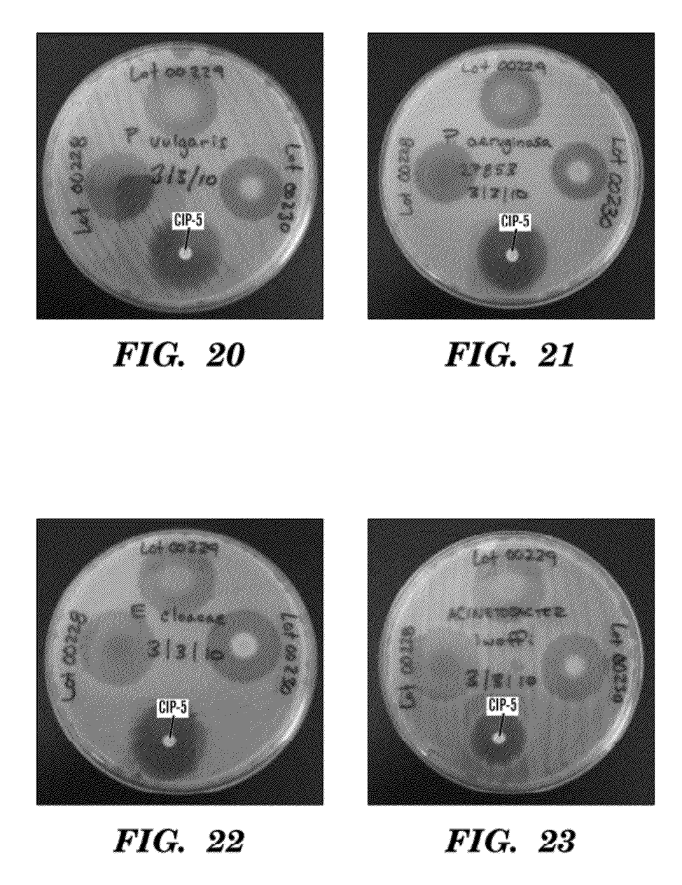

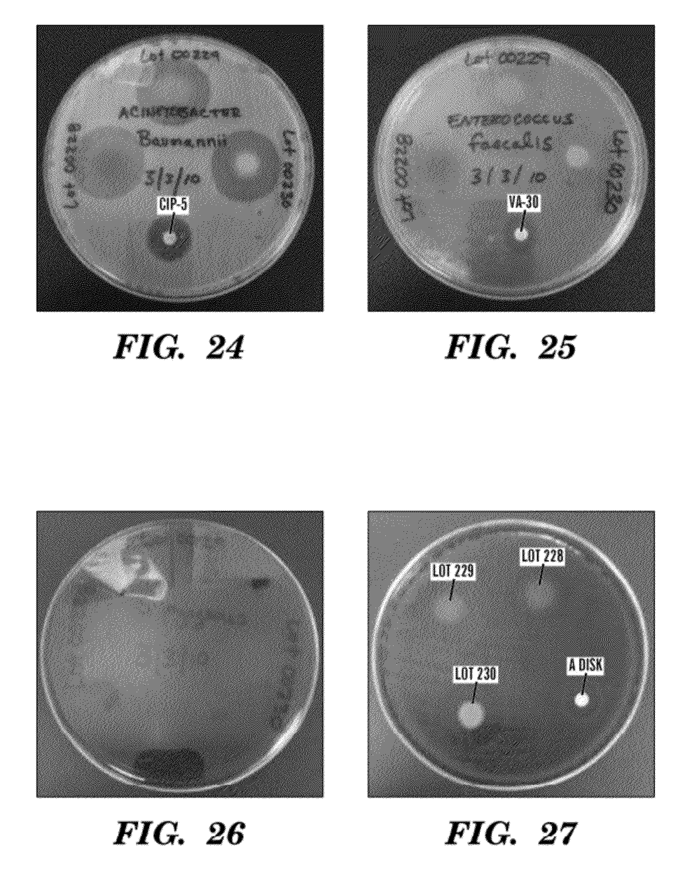

This is a consistent result for each Lot dose as shown against Staph aureus (FIG. 16), Methicillin Resistant Staph Aureus (MRSA) (FIG. 17), Klebsiella pneumoniae (FIG. 18), E. coli (FIG. 19), Proteus vulgaris (FIG. 20), Pseudomonas aeruginosa (FIG. 21), Enterobacter cloacae (FIG. 22), Acinetobacter lwoffi (FIG. 23), Acinetobacter baumanii (FIG. 24), Enterococcus faecalis (FIG. 25), Streptococcus pyogenes shown in front view (FIG. 26) and back view (FIG. 27), and a presumptive test for Group-A Strep, shown in the back view of FIG. 27 (using Lot 230 only). Of interest is the addition of identical size drops (i.e. diameters of ZOA) of alternative active pharmaceutical agents as a control. These show considerably reduced zones of inhibition that the formulations of the invention, as shown in: FIG. 16, in which CIP-5 is Ciprofloxacin, E-15 is Erythromycin, and Clindamycin; FIG. 17, in which E-15 is Erythromycin, CC-2 is Clindamycin, CIP-5 is Ciprofloxacin, and Fox-10 is Cefoxicillin; FIGS. 18-24, in which CIP-5 is Ciprofloxacin; and FIG. 25, in which VA-30 is Vancomycin.

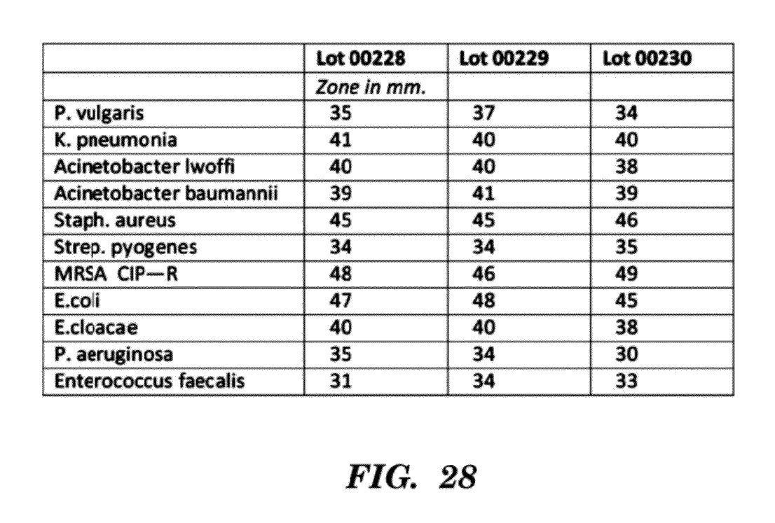

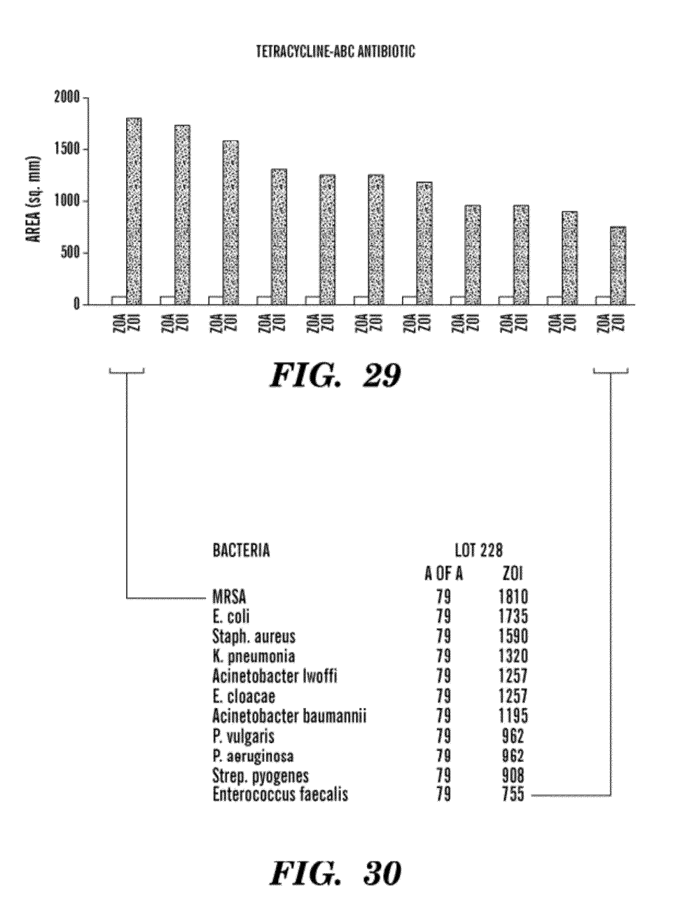

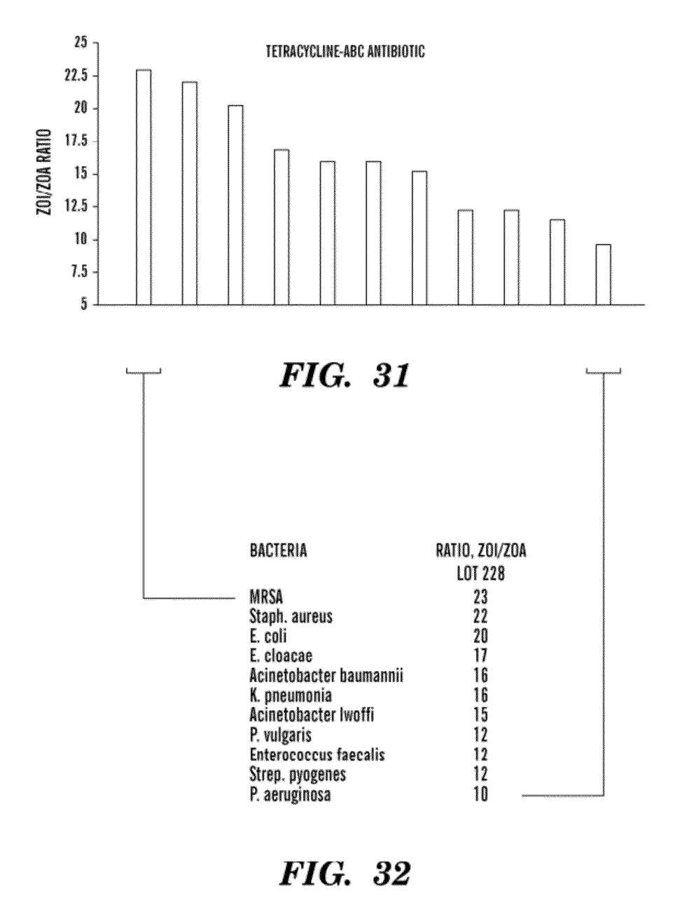

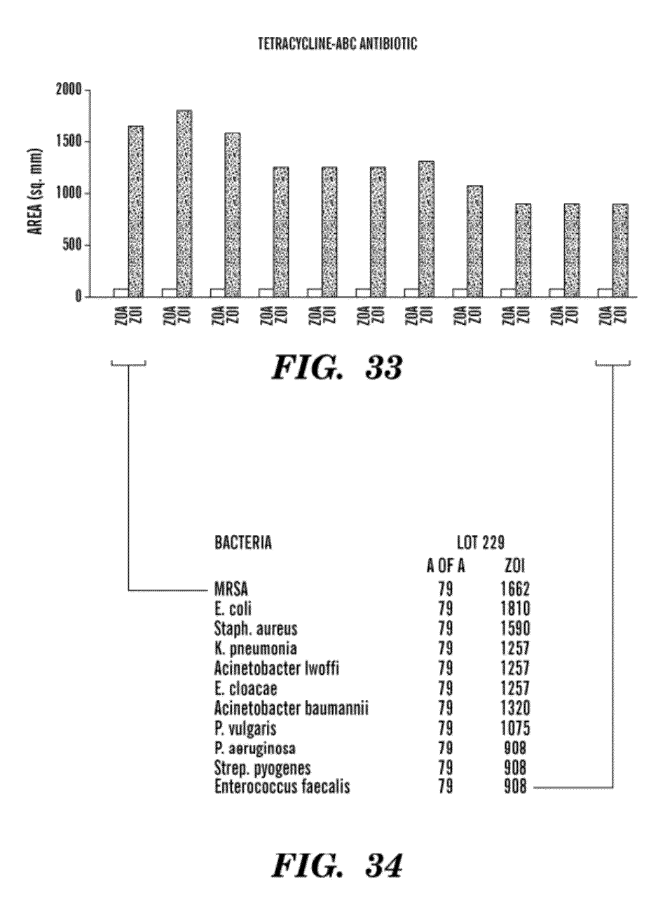

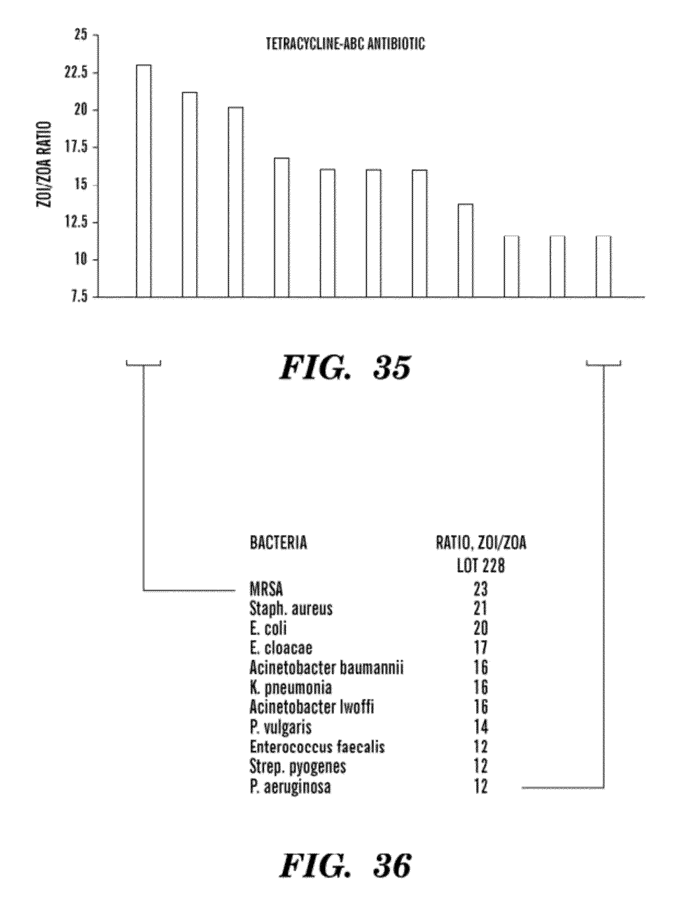

One conclusion from these tests is that the amplified or expanded area forming the Zone of Inhibition available with the inventive formulations ranges from 10 times to 23 times the area of application of the antibiotic. Additionally, diffusion and spreading is enhanced by the use of the selected chemical stabilizer when used as an ingredient in the formulation of TCN with double carriers. Again, this effect was noted for every bacterial culture for which test data is available. For spreading drugs from the skin or other site of pathogen load into adjacent tissue, this effect can be beneficial. Also, the efficacy of the primary therapeutic effect of this antibiotic was unchanged with the addition of various amounts of the chosen stabilization agent. FIG. 28 is a summary compilation of the zones of inhibition for the data shown in FIGS. 16-27. FIG. 29 graphs (with a reference legend at FIG. 30) the Lot 00228 comparison of the zones of inhibition areas in square millimeters to the zones of application areas. In similar manner, FIG. 31 graphs (with reference legend at FIG. 32) the ratios of ZOI/ZOA from Lot 00228. This shows that the amplified biological coverage that is achieved with an embodiment of the invention ranges from 10 to 23 times the area of application of the active agent. FIGS. 33-36 graph the comparable data from Lot 00229, which shows ranges that the amplified biological coverage that is achieved with that embodiment of the invention ranges from 12-23 times the area of application of the active agent.

What is observed is that diffusion and spreading of the active agent is enhanced by the use of a stabilizer, particularly at relatively higher amounts. The stabilizer's (i.e., dispersant's or anti-oxidant's) characteristic of achieving a substantially homogeneous distribution of active agent ensures uniform primary therapeutic effect at sites of application to tissue. It also mitigates the likelihood of pockets of higher concentration of active agent (also known as “peaks”) which are generally undesirable. A further criticality was discovered in these inventive embodiments by which this favorable homogeneous distribution is adversely affected by use of substantial amounts of a polar aprotic solvent as one of the penetration enhancers. In this regard, the homogeneity is degraded as the amount of use of that solvent class, as represented by a DMSO type of ingredient, trends toward at least about 50% of the therapeutic drug delivery composition. This was also noted in the dose ranging tests 17 and 18 herein below, each having a higher than desired DMSO amount according to this invention, and an ensuing loss of active agent homogeneous distribution. This is yet another discovered criticality in maintaining that type of ingredient at a very low percent of the overall composition.

A further observation of these experiments is to confirm that the formulations remain fully effective or biocidal as higher amounts of anti-oxidant is added. This discovery is further useful in the intentional design according to the goals of the invention so that additional high level anti-oxidant secondary therapeutic effect is achievable without degrading the primary therapeutic effect of the active agent.

The amplified biological coverage achieved in these tests yields a ZOI:ZOA ratio of no less than 5× for all bacteria tested—and with a ratio of 10×-20× for most bacteria. Moreover, the range of bacteria tested, both in these experiments and in human field studies, establish these embodiments of the invention with tetracycline as being remarkably broad spectrum high-efficacy pharmaceutical compositions.

Dose Ranging Studies

Additional laboratory testing of various embodiments of the invention was conducted against various pathogens. In each test sample, the AAneed data is approximately the amount of stabilizer needed to scavenge 50% of oxygen free radicals. The following formulations were tested:

### Sample #: 1

0.10%=TET concentration by weight (%)
3.0%=AA concentration by weight (%)
2.4%=DMSO concentration by weight (%)
94.50%=DPG concentration by weight (%)

### Color and Chemical Stability Info

### 420=Ratio of AAhere/AAneed

### Sample #: 2

0.30%=TET concentration by weight (%)
3.0%=AA concentration by weight (%)
2.6%=DMSO concentration by weight (%)
94.06%=DPG concentration by weight (%)

### Color and Chemical Stability Info

### 420=Ratio of AAhere/AAneed

Sample #: 3

1.00%=TET concentration by weight (%)
3.0%=AA concentration by weight (%)
3.5%=DMSO concentration by weight (%)
92.51%=DPG concentration by weight (%)

### Color and Chemical Stability Info

### 421=Ratio of AAhere/AAneed

### Sample #: 4

3.00%=TET concentration by weight (%)
3.0%=AA concentration by weight (%)
5.9%=DMSO concentration by weight (%)
88.10%=DPG concentration by weight (%)

### Color and Chemical Stability Info

### 423=Ratio of AAhere/AAneed

Sample #: 5

0.10%=TET concentration by weight (%)
1.0%=AA concentration by weight (%)
0.9%=DMSO concentration by weight (%)
98.02%=DPG concentration by weight (%)

### Color and Chemical Stability Info

### 138=Ratio of AAhere/AAneed

### Sample #: 6

0.30%=TET concentration by weight (%)
1.0%=AA concentration by weight (%)
1.1%=DMSO concentration by weight (%)
97.58%=DPG concentration by weight (%)

### Color and Chemical Stability Info

### 138=Ratio of AAhere/AAneed

### Sample #: 7

1.00%=TET concentration by weight (%)
1.0%=AA concentration by weight (%)
2.0%=DMSO concentration by weight (%)
96.03%=DPG concentration by weight (%)

### Color and Chemical Stability Info

### 139=Ratio of AAhere/AAneed

### Sample #: 8

3.00%=TET concentration by weight (%)
1.0%=AA concentration by weight (%)
4.4%=DMSO concentration by weight (%)
91.62%=DPG concentration by weight (%)

### Color and Chemical Stability Info

### 140=Ratio of AAhere/AAneed

### Sample #: 9

0.10%=TET concentration by weight (%)
0.3%=AA concentration by weight (%)
0.3%=DMSO concentration by weight (%)
99.25%=DPG concentration by weight (%)

### Color and Chemical Stability Info

### 41=Ratio of AAhere/AAneed

### Sample #: 10

0.30%=TET concentration by weight (%)
0.3%=AA concentration by weight (%)
0.6%=DMSO concentration by weight (%)
98.81%=DPG concentration by weight (%)

### Color and Chemical Stability Info

### 41=Ratio of AAhere/AAneed

### Sample #: 11

1.00%=TET concentration by weight (%)
0.3%=AA concentration by weight (%)
1.4%=DMSO concentration by weight (%)
97.27%=DPG concentration by weight (%)

### Color and Chemical Stability Info

### 41=Ratio of AAhere/AAneed

### Sample #: 12

3.00%=TET concentration by weight (%)
0.3%=AA concentration by weight (%)
3.8%=DMSO concentration by weight (%)
92.86%=DPG concentration by weight (%)

### Color and Chemical Stability Info

### 42=Ratio of AAhere/AAneed

### Sample #: 13

0.10%=TET concentration by weight (%)
0.1%=AA concentration by weight (%)
0.2%=DMSO concentration by weight (%)
99.60%=DPG concentration by weight (%)

### Color and Chemical Stability Info

### 14=Ratio of AAhere/AAneed

### Sample #: 14

0.30%=TET concentration by weight (%)
0.1%=AA concentration by weight (%)
0.4%=DMSO concentration by weight (%)
99.16%=DPG concentration by weight (%)

### Color and Chemical Stability Info

### 14=Ratio of AAhere/AAneed

### Sample #: 15

1.00%=TET concentration by weight (%)
0.1%=AA concentration by weight (%)
1.3%=DMSO concentration by weight (%)
97.62%=DPG concentration by weight (%)

### Color and Chemical Stability Info

### 14=Ratio of AAhere/AAneed

### Sample #: 16

3.00%=TET concentration by weight (%)
0.1%=AA concentration by weight (%)
3.7%=DMSO concentration by weight (%)
93.21%=DPG concentration by weight (%)

### Color and Chemical Stability Info

### 14=Ratio of AAhere/AAneed

### Sample #: 17

3.00%=TET concentration by weight (%)
3.0%=AA concentration by weight (%)
50.2%=DMSO concentration by weight (%)
43.82%=DPG concentration by weight (%)

### Color and Chemical Stability Info

### 412=Ratio of AAhere/AAneed

### Sample #: 18

3.00%=TET concentration by weight (%)
0.3%=AA concentration by weight (%)
51.5%=DMSO concentration by weight (%)
45.24%=DPG concentration by weight (%)

### Color and Chemical Stability Info

### 40=Ratio of AAhere/AAneed

Clinical Evaluations

Numerous human field studies have been successfully performed using embodiments of the invention according to clinical need.

Study I:

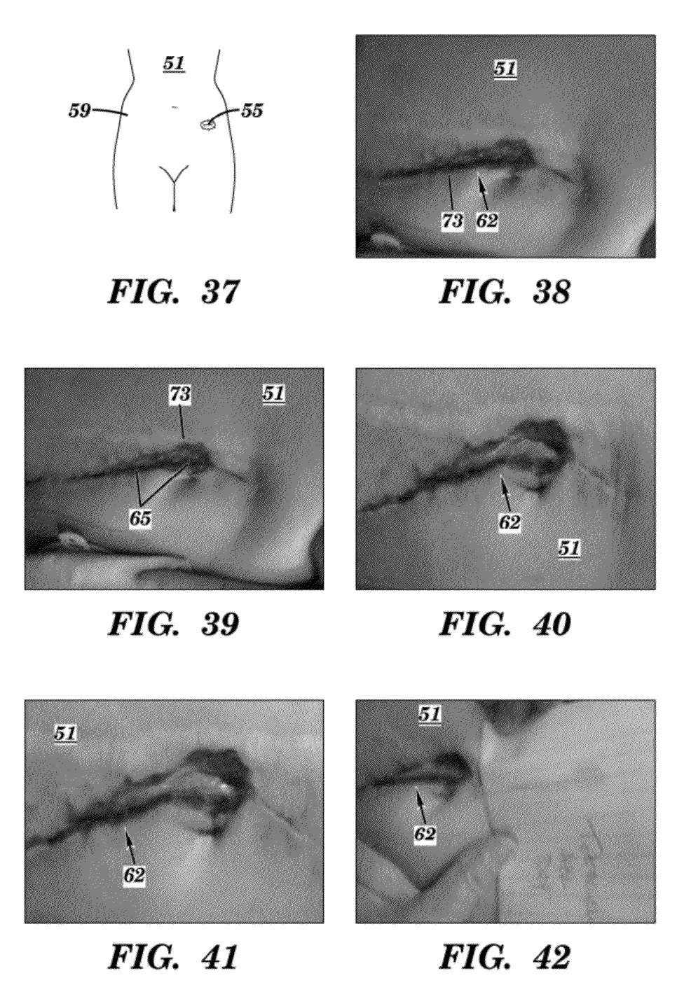

The following summary of a field study of a colostomy patient who developed a no recourse staph infection in a hospital is provided, with reference to FIGS. 37-42. The patient 51 had colostomy surgery on Feb. 3, 2010, in Oklahoma City, and follow-up care at the same location. Care at home was then provided to the patient by a registered nurse (RN). A colostomy consists of an artificial opening 55 (stoma) created in the large intestine 59 and brought to the surface of the abdomen for the purpose of evacuating the bowels. The aim of the colostomy is to restore the outflow of feces from a location in the intestine above an area that is healing or which has been surgically removed. The normal healing process became complicated when a bacterial infection developed at the surgical site 62 about one week after the surgery. The infection was localized to an area the patient called the “bad spot” as indicated by inflamed red areas 73 surrounding the incision. The infection developed a puss discharge and excessive drainage. Medical history of relevance includes: female, age 58, smoker, non-diabetic, no slow-healing history; allergic to some antibiotics.

Thu 1/28: Received pre-surgical flagyl, 500 mg/dose (Q12), cipro, 500 mg/dose (Q12), and gentamycin, dose not noted (Q6), all IV. These were continued daily until all three meds were discontinued on Feb. 5, 2010.

Wed 2/3: Surgery was on this day. Patient had previous allergic reactions to penicillin and predicted allergic reaction to ampicillin. Post-surgical meds included flagyl, 500 mg/dose (Q12), cipro, 500 mg/dose (Q12), and gentamycin, dose not noted (Q6), all IV. These were continued daily until all three meds were discontinued on Feb. 5, 2010.

Fri 2/5: Detection of a Strep-A strain, confirmed by culture study. Shifted from gentamycin to vancomycin 1.5 g BID, Q12. Patient was kept on this dosing regimen until 2/8 (date of discharge from hospital).

Mon 2/8: Patient received the last of seven doses of vancomycin. Throughout all of this her peaks (within 1 hour of receiving vancomycin) and troughs (before receiving vancomycin) were normal (ranges not noted here). Patient was discharged from the hospital on this date.

Wed 2/10: The normal healing process became complicated when a bacterial infection developed at the surgical site, now one week after the surgery. This was possibly a progression of the previously diagnosed Strep-A bacterial infection; but could have been complemented by a secondary bacterial infection. The infection was localized to the area she called the “bad spot.” The infection developed a puss discharge and excessive drainage.

Fri 2/12: Patient's incision was now dehiscing and draining about 4-5 tablespoons of infected material each day. Initial response was to apply Dakins solution, but it did not dry out the incision. RN called doctor's office and spoke with Resident. No functional assistance rendered. Mon 2/15: RN telephoned doctor's office and spoke with the surgeon who had performed the surgery. No functional assistance rendered, and no recommendation available for an effective medication. Therefore medical authority was granted to use an experimental formulation of the tissue penetrating topical drug delivery system of the invention, using tetracycline as the active pharmaceutical agent. Patient consent was properly obtained.

Wed 2/17: Due to the Patient having a known allergy to some antibiotics, an allergy test was done to establish that she was not allergic to tetracycline and the drug delivery system components. This test was done one day prior to use of the antibiotic on the wound infection. At the actual site of the infection, there was considerable inflammation and red areas 73 surrounding the incision 62. Considerable puss discharge 65 was occurring from the surgical incision.

Thu 2/18: Referred to as “Day Zero”, this was the day the Patient's nurse began using one of the embodiments of this invention on the wound infection, which was believed to be a deep tissue Staph-A infection. This was now two weeks after the surgery. Given that no alternative treatment was available, and by medical direction, the RN commenced administration of one drop of the drug delivery solution at the top of the wound and allowed it to roll down the entire length of the wound. RN then massaged the solution into the tissue with a Q-Tip at the wound site. This was performed three times a day (Q8). Patient was advised that the drug delivery solution was expected to be fast acting, and that results should be seen within 3 days.

Fri 2/19: “Day 1” RN administered same therapy as 2/18. Patient was “up and about” and went out to dinner. Patient reported that the “bad spot” was looking better and that the puss drainage had nearly stopped. RN confirmed an improved visual change to site of treatment.

Sat 2/20: “Day 2” RN administered same therapy as 2/18 and 2/19, with similar improvements noted. Puss generation reduced to almost zero, as shown in FIG. 41.

Sun 2/21 “Day 3” RN administered same therapy as 2/18, 2/19 and 2/20. The “bad spot” was, in the Patient's words, “looking better every day.” She reported that the puss drainage had fully stopped on Day 3, and the drainage from that site was less and less, and that it looked cleaner and cleaner with each passing day. The tentative conclusion was that the bacterial infection had been completely resolved with a 3-day treatment of the Tetracycline-based tissue penetrating drug delivery system of the invention. Although both the Patient and the RN shared these observations and conclusions, the opinion of a physician was obtained for final confirmation.

Mon 2/22: “Day 4” Patient returned to her physician for a checkup. A close inspection of the surgical wound area resulted in the physician saying that it was “just fine,” and he saw no reason to treat her with any alternative agents for the now-resolved bacterial infection. The “bad spot” area was observed to now be healing from the inside out, and healing progress was judged to be good. Same therapy as 2/18 was continued, but used the new antibiotic formulation only twice daily in view of physician observations.

Tue 2/23: “Day 5” Same therapy as 2/18 but only twice daily. No stinging or tingling was reported as a result of the application of the medication. Patient reported that the color of the tissue in the area of the prior wound infection had progressed from a red (inflamed) color to a more pink (healthy) color as shown in FIG. 42. When asked for a report of the tingling or stinging when using the new antibiotic formulation during Day 0 through Day 5, the Patient's answer was, “it never did burn or sting at all, even from the first time we used it.” When asked for a numerical evaluation on a sensitivity scale (zero=water; 10=alcohol sting), Patient said, “Either a zero or a one; I really couldn't feel any sting at all.”

Wed 2/24: “Day 6” Same therapy as 2/18 but only twice daily.

Thu 2/25: “Day 7” Same therapy as 2/18 but only twice daily. Wound site no longer red or inflamed. Excellent granulation. Very minimal drainage.

Thu 2/26: “Day 8” Same therapy as 2/18 but only twice daily.

Tue 3/9: Follow up visit two weeks later. All was well. Underlying healing from the surgery was progressing nicely, and as expected, based on follow-up visit with the Patient and her nurse. No additional bacterial infections had developed. The deep tissue Staph-A bacterial infection had been defeated by use of one embodiment of the medication and drug delivery system of the present invention.

Study II:

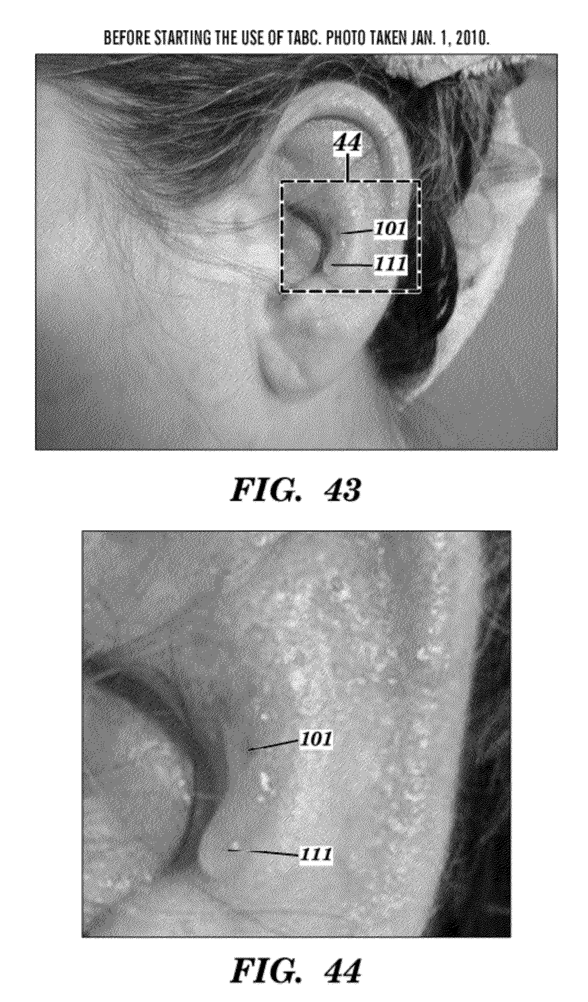

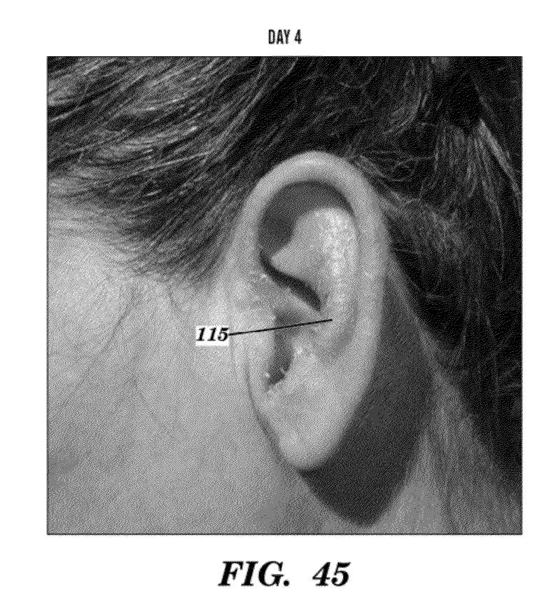

Referring to FIGS. 43-44, there is shown a patient suffering from a MRSA-infected ear lesion 101. This patient had extensive bodily sites of active MRSA infections on the chest, lower spine, lower abdomen, and other locations. Her misery was substantial and she had endured months of failed pharmaceutical treatments, while becoming progressively socially isolated and with increased despair. As is common, the methicillin resistant staph aureus infections weep a distinctive fluid 111 from sites of lesions, such as lesion 101. This phenomenon contributes to the challenges for such patients.

Upon receipt of patient and physician consent, a tetracycline medicament embodiment according to the invention was provided. This embodiment comprised less than about 20% DMSO and more than about 80% dipropylene glycol, as well as ascorbic acid. The patient applied the medication 18 times over a four day period. On day four of the treatment, the infected site demonstrated healthy pink tissue 115, as shown in FIG. 45. Other sites were also demonstrating rapid improvement through the killing of the pathogens and restoration of healthy tissue sites. The patient was overjoyed that a solution had been found. Long-term follow up has been positive.

Study III:

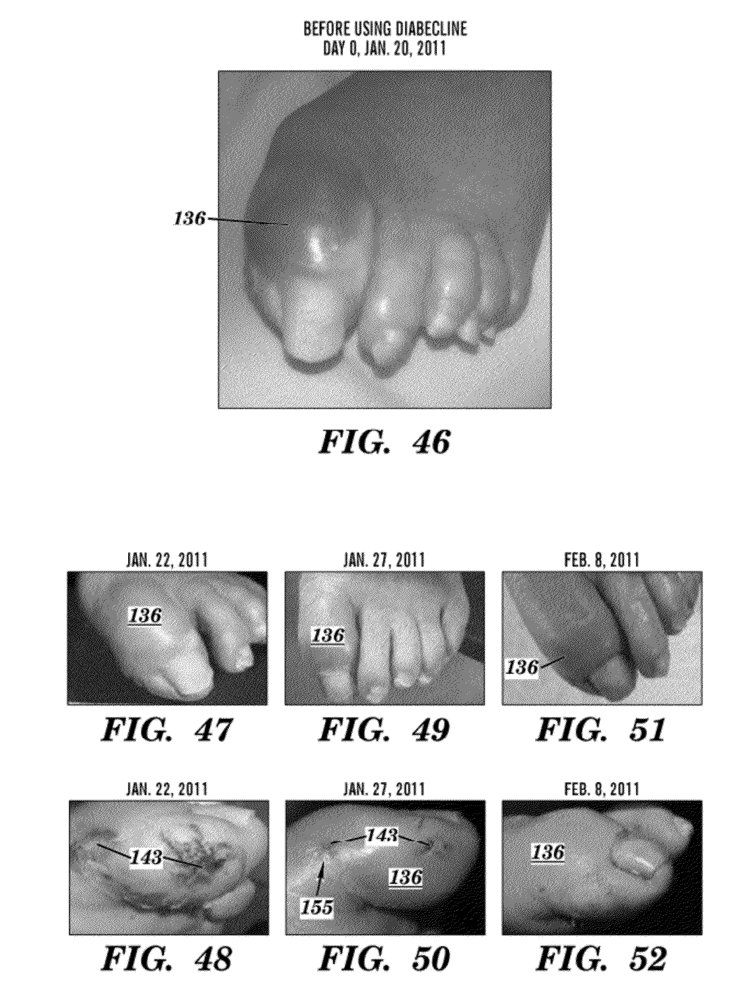

FIGS. 46-52 relate to serious diabetic foot lesions treated in yet another human field study of an embodiment of the invention. In this embodiment, the additional ingredient to hasten a third therapeutic tissue healing effect was added. This embodiment used vitamin D in a small but medically efficacious amount. Referring to FIG. 46, a 76 year old diabetic woman residing in a nursing home presented with a severely swollen left great toe 136. She was admitted to a hospital and diagnosed with cellulitus. She was placed on IV vancomycin for MRSA, based on a positive blood culture and a soft tissue culture. The patient did not respond to the vancomycin treatment and a podiatrist was called into the case 8 days following admission to assess care options for the lesions 143 on the medial great toe and the ongoing degradation caused by the inflamed condition.

After appropriate consent, the podiatrist commenced therapy using the above described embodiment of the invention. As seen in the medial view of FIG. 48, after two days of treatment, the wound had improved remarkably. The dark red appearance had subsided and the swelling associated with cellulitus had decreased about 60%. In FIG. 48, the red area near the base of the toe shows an underlying ulcer that was not even apparent on day 0 or day 1 of the present treatment due to edema. The ulcerated portion in the distal front aspect of the toe was debrided on day 0, with remaining tissue appearing healthy. The foul odor of the toe had subsided with two days of treatment using the invention.

FIGS. 49 and 50 show the toe after seven days of treatment. The toe 136 is healing at an unprecedented rate for this type of patient. The edema has subsided about 80% when compared with day 0, highly suggestive of resolution of the cellulitus. Indeed, the bright red color associated with cellulitus has virtually completely gone and the toe has the color of the other toes, as best seen in FIG. 49. The infected edematous top layer of skin has now taken on a dried flaky nature enabling easy debridement with a simple shedding or peeling maneuver. The underlying skin is shown in FIG. 50 as clean and feeling soft to the touch. Indeed, the skin had the consistency of baby skin, and looked much younger than the rest of the foot that was not infected. Debridement revealed the small ulcer 155 at the base of the toe which was probably the portal of entry for the staph causing bacteria that led to her cellulitus and subsequent diagnosis of a MRSA infection. Again, all evidence of a foul odor was completely absent.

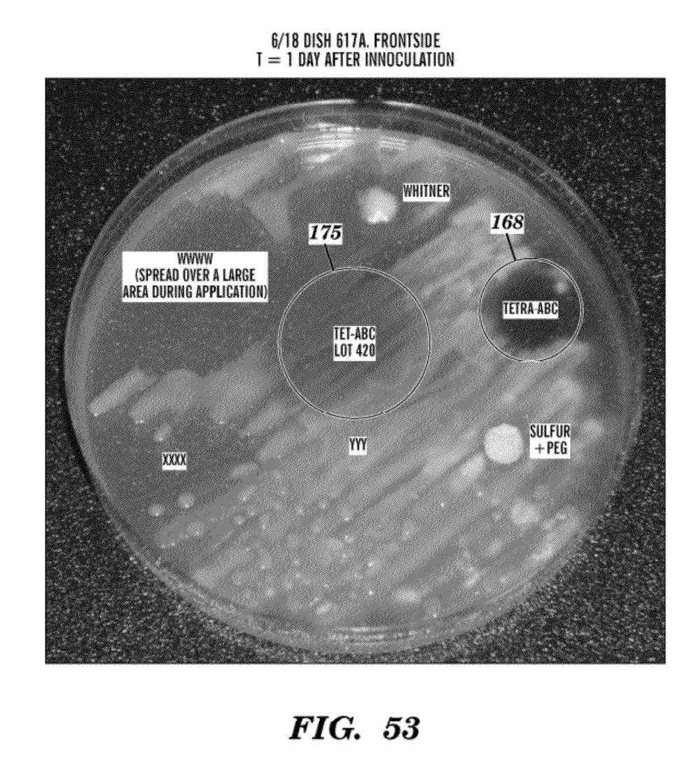

FIGS. 51 and 52 show the previously MRSA-infected toe 136 at the 20 day mark of treatment with the medication as described herein. The edema has completely resolved and the toe is now its normal size and shape. It is again noted that the skin looks healthier than the toes that were not treated. Referring to the dorsal view of FIG. 51, the thin, shiny nature of the other toes not treated is a sign of poor circulation common with diabetics. However, the big toe that was treated does not have that appearance, indicating that the circulation has been improved as a consequence of treatment. As seen in FIG. 52, the infection has completely resolved, the original offending ulcer has closed and can longer be located due to healthy tissue replacement. On day 20 the treatment was discontinued since the patient was healed from the MRSA infection and Grade I diabetic ulcer of the left hallux with cellulitus that extended to midfoot.

Study IV:

FIG. 53 is a top view of a Petri dish inoculated with a common strain of a yellow toenail fungus obtained from a volunteer, and several experimental active agents and delivery systems after one day. Of interest to this application is circle 168, which outlines the location of a tetracycline embodiment of the invention, with a gel-barrier style drug delivery ointment as described and claimed herein. The dark area within the circle is the zone of application and the zone of inhibition. Notably, the drug delivery reservoir effect of the antibiotic embodiment shown creates a constant-source diffusion that functions, medicinally, as a potent anti-fungal agent. In contrast, the non-reservoir embodiment shown at circle 175 was demonstrably less efficacious than the reservoir embodiment.

Study V:

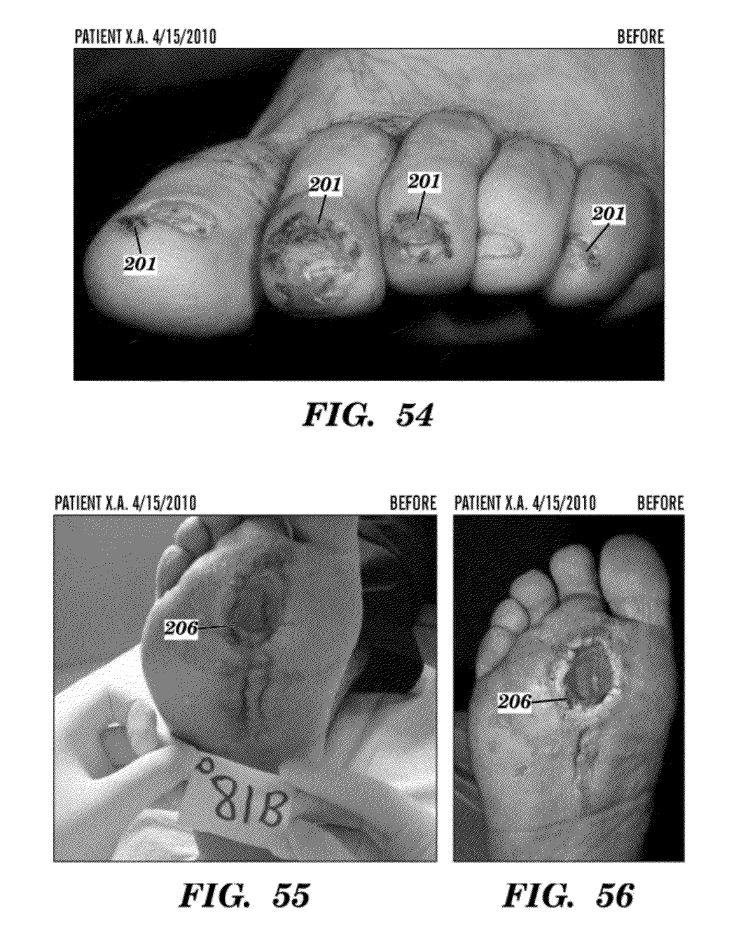

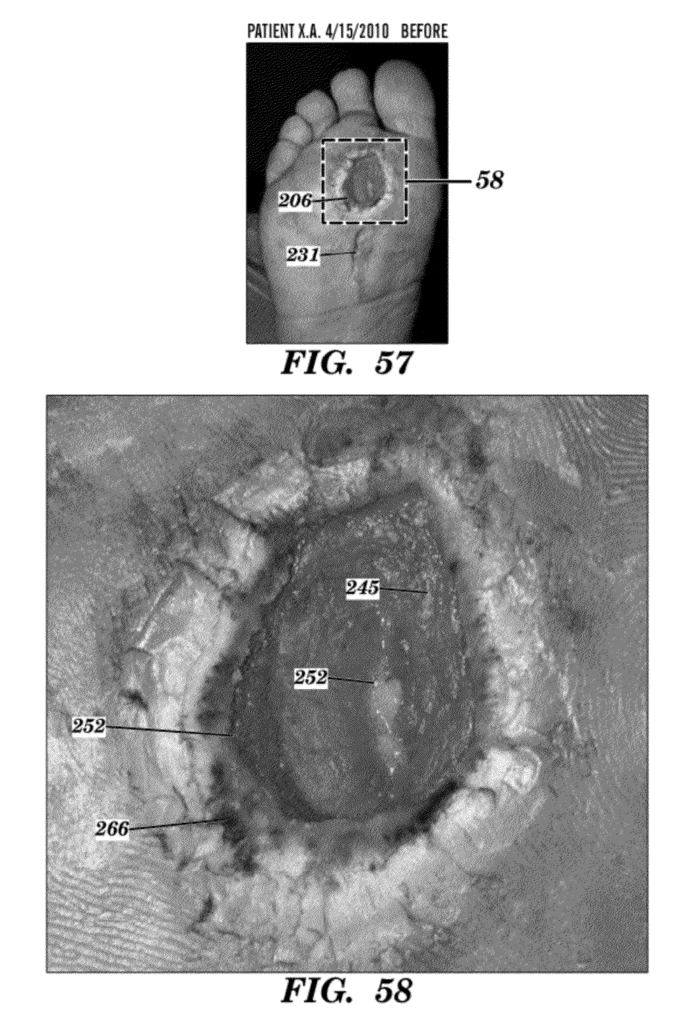

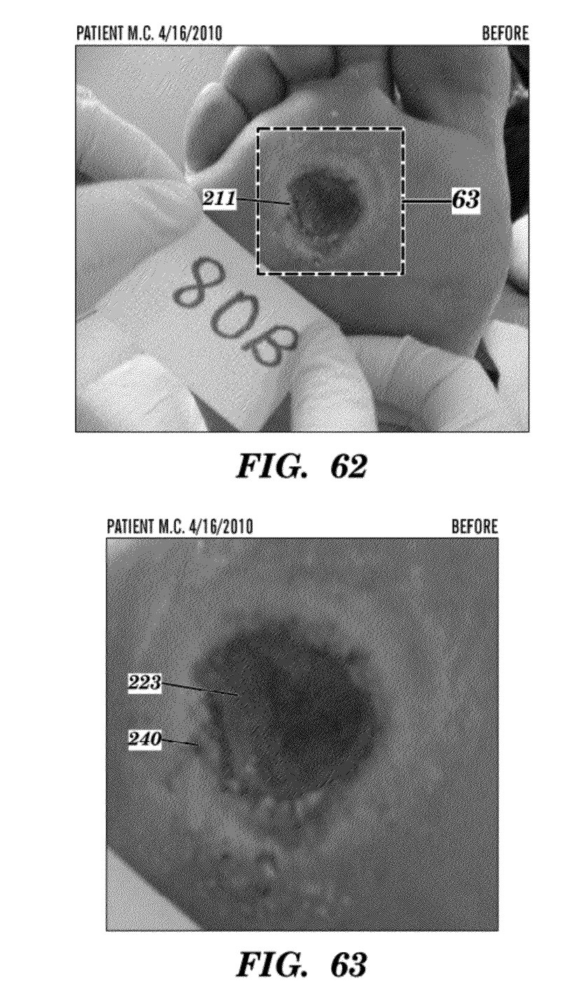

Referring to FIGS. 54-64, a further human clinical field study was performed on diabetic foot patients at a diabetic wound clinic. Patient 81B, or X.A., is shown with lower extremity diabetic wounds 201 in FIGS. 54 and 206 in FIGS. 55-56. The patient had received aggressive medical treatment for a period of time, but improvement was not forthcoming. FIGS. 56 and 57 show the extent of tissue breakdown on the patient's plantar base. The probability of eventual amputation was set at about 60% prior to use of the treatment of the invention. The patient was selected for experimental treatment as a last resort because of no improvement from prior treatments. FIG. 58 is a close-up view of a portion of FIG. 57, and show a prior wound 231 that is only partially healed, subcutaneous layers 245 of muscle, areas 252 of poor vascular supply, and some necrotic tissue formation 266.

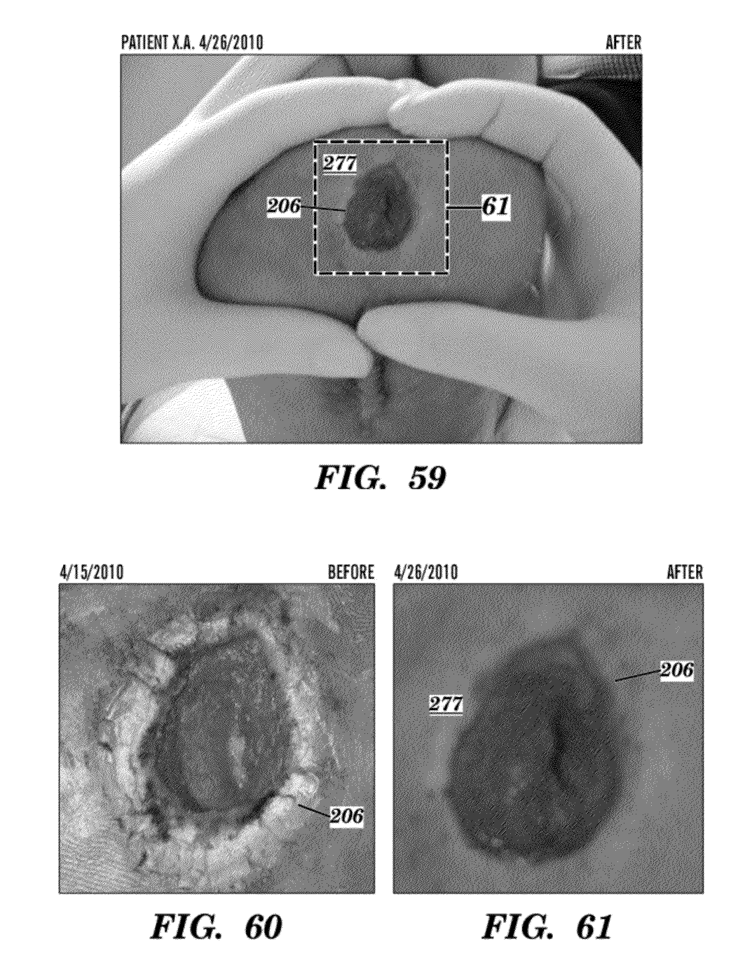

Treatment of Patient X.A. was begun with a 3% tetracycline and vitamin D embodiment disclosed herein. FIGS. 59 and 61 show the progress of the wounds after 11 days of treatment, and as compared with FIG. 60 showing the pre-treatment identical site. The granulation 277 was proceeding consistent with rapid healing. The yellow color is the medication. The remarkable improvements led to a medical team decision of not amputating the foot and continuing with the successful therapy of the invention.

Study VI:

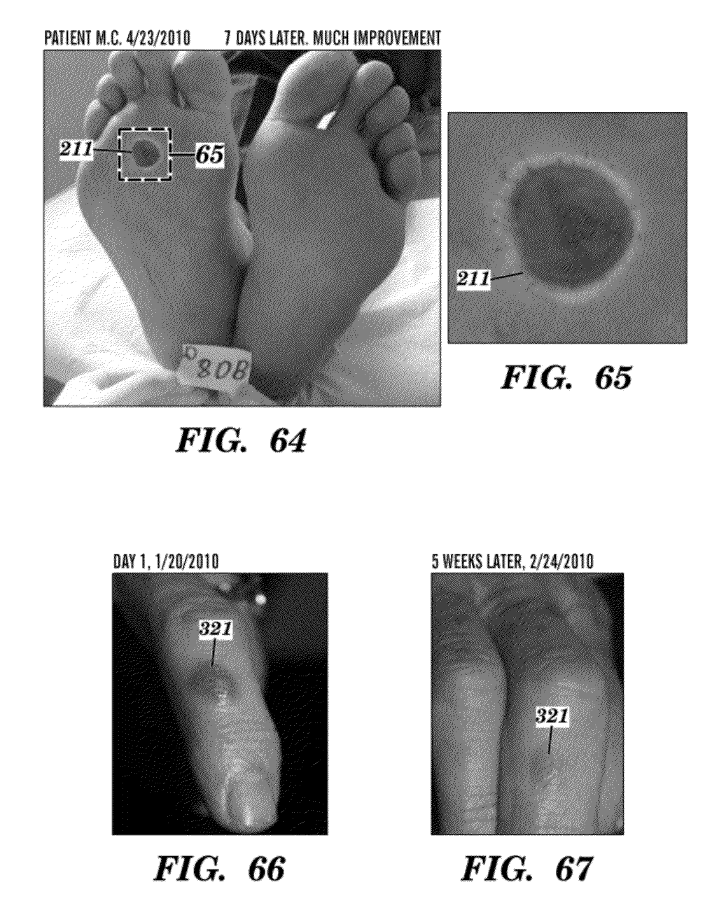

A second patient, M.C. or 80B, was selected for treatment and evaluation. Similar to patient 81B, this patient had received aggressive but unsuccessful prior care. Referring to the close-up view of FIG. 63, the lesion 211 displayed deep subcutaneous layers 223 of muscle, and necrotic tissue sites 240. FIG. 64 and close-up FIG. 65 show the lesion 211 after seven days of treatment according to the invention. Granulation was proceeding consistent with rapid healing. The vascular supply was improved, based on visual observation and the uniform color of the underlying tissue. All signs of infection had gone. These results confirmed and validated the efficacy of the tetracycline and vitamin D embodiment of the invention as a treatment of choice for diabetic wounds and lesions.

Study VII:

FIG. 66 discloses a pre-treatment view of a suspected spider bite 321, displaying redness and discharge of pus indicative of a rapid onset infection. On day 1, the patient commenced use of an embodiment of the invention to avoid the development of cellulitus and serious infection, both of which are common with such bites. Continued improvement occurred, so that by the fifth day of treatment the lesion had dried up and there was no pus discharge or infection. The redness and inflammation had ceased, granulation had occurred, and treatment was stopped. FIG. 67 shows the site of the bite five weeks following the treatment with excellent long-term health of the tissue. A noted advantage of users of the invention of all wounds is an analgesic affect. This is particularly important and useful for painful injuries such as brown recluse spider bites which are notoriously painful in a short amount of time.

Study VIII:

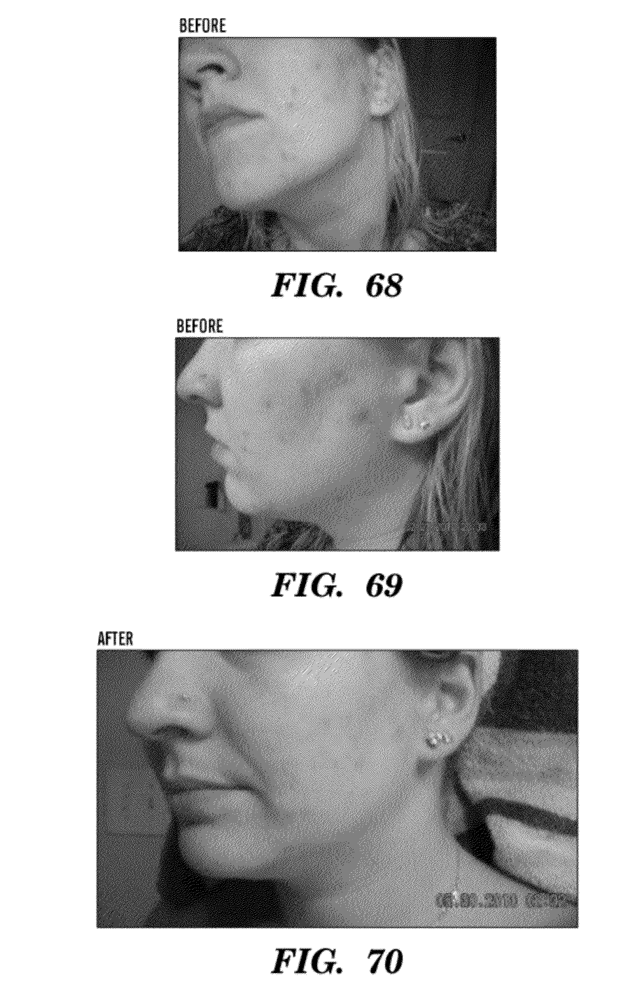

FIGS. 68 and 69 show a side of the face of a patient with a history of intransigent acne. Following appropriate consent, the patient was provided with a 3% tetracycline embodiment of the invention. The patient used the composition for three weeks on the side of the face shown in FIGS. 68-69, with evident improvement. The lesions that existed prior to the treatment faded or disappeared, and no new lesions appeared during the use of the therapeutic ointment. FIG. 70 shows the result after the three week use of this embodiment of the invention.

Study IX:

An embodiment of the invention was applied to a young male's forearm after experiencing a psoriasis flareup. After only one day of use, the skin condition had markedly improved. After two days of treatment, there was dramatic reduction in the appearance of the previously swollen red patches of skin, and a complete reduction of the silvery flaky detritus had occurred.

Study X (in Process):

It is recognized that embodiments of the invention provide exceptional deep tissue penetration of the active pharmaceutical agent. Indeed, the molecular weights of the constituent elements of the drug delivery system have been selected to enable penetration through the cellular walls of numerous pathogens. In this manner, the drug delivery system is able to carry the pharmaceutical active agent to cellular sites of pathogens in a manner not previously accomplished. This is important with respect to highly resistant pathogens, such as those noted herein, and to medical scenarios in which traditional treatment modalities are inadequate. An example of this scenario is a high velocity trauma wound, such as from a military rifle or explosive, although such wounds can be caused by industrial, farm or automobile accidents as well. In this example, pathogen-laden fragments are dispersed throughout a vast wound bed. It is impossible for initial or subsequent lavage to clean the wound fully. Consequently these wounds are commonly the sites of subsequent tenacious infections that exhibit periodic rebounds and colony growth. The antibiotic medication, using a preferred deep tissue penetrating drug delivery system of the invention, enables highly effective delivery of medication to all sites of such a wound where pathogenic material may reside. This yields exceptional kill capability to otherwise ineffective traditional medications, and excellent wound recovery where little hope existed prior to use of the present inventions. This embodiment may be useful as surgical wash or lavage following initial debridement and standard lavage according to the status of the wound bed. One embodiment of the invention is, therefore, a surgical medicament for use as a penetrating medicated lavage in a deep tissue wound. This medicament is formed as a multi-functional solution, suitable for delivering at least one active pharmaceutical ingredient to desired locations of mammalian host tissue for primary therapeutic effect against bacterial pathogens at the desired locations and adjacent surgically inaccessible locations. The medicament is also designed for delivering at least one secondary therapeutic effect by weakening the pathogen survival systems against the at least one active pharmaceutical ingredient thereby enhancing the primary effect of the active pharmaceutical ingredient and by improving healthy tissue natural response mechanisms in tissue adjacent to the pathogens. The medicament comprises a non-hygroscopic first chemical penetration enhancer, a hygroscopic second chemical penetration enhancer, an anti-oxidizing dispersant, and an active pharmaceutical ingredient. The minimum number of ingredients is particularly valuable in minimizing adverse reactions in larger wound sites.

In this embodiment, the non-hygroscopic first chemical penetration enhancer has solvent properties suitable for solubilizing an active pharmaceutical ingredient, and has a first diffusion constant suitable for carrying the solubilized active pharmaceutical ingredient through mammalian skin and other tissue to pathogen locations in that skin and tissue to achieve primary therapeutic effect against the pathogens. The first chemical penetration enhancer further has characteristics suitable for carrying the active pharmaceutical ingredient through the cell walls of pathogens to deliver a portion of active pharmaceutical ingredient to an interior portion of the pathogen within the cell wall thereby enhancing the primary therapeutic effect of the active pharmaceutical ingredient against the pathogens. Also the first chemical penetration enhancer has a percent range in the medicament of between about 2% and 15%.

The hygroscopic second chemical penetration enhancer in this embodiment has diluent properties for diluting the first chemical penetration enhancer and an active pharmaceutical in solution to optimize the solution for mammalian tissue compatibility, particularly in such large wound beds. Importantly, it has further characteristics for providing a zone of enhanced inhibition to provide protection from any pathogenic effect between the adjacent healthy tissue and the pathogens. The second chemical penetration enhancer preferably has a percent range in the medicament of between about 98% and 85%; and it has a second diffusion constant that is different than the diffusion constant of the first penetration enhancer;

The anti-oxidizing dispersant is mixable in solution with the first and second chemical penetration enhancers and the active pharmaceutical ingredient. The dispersant is in a percent of the medicament of between 3% and 10% and is suitable for providing multiple secondary therapeutic effects by interaction with the active pharmaceutical ingredient to ensure maintenance of substantial homogeneous distribution of the selected active pharmaceutical ingredient in the medicament during delivery to all areas of the wound bed and adjacent tissue, and it further reduces the water activity level of the medicament to cause water stress in any pathogen contacted by the medicament. However, the medicament causes only temporary reversible water level reduction in adjacent host tissue. Finally, an active pharmaceutical ingredient is present in the medicament in an amount from about 0.1% to about 5% the medicament. The medicament may be selected from a variety of active agents, though at two of higher interest may be tetracycline and an aminoglycoside, such as tobramycin sulfate or similar agent.

### Additional Embodiments

Accordingly, the inventions have excellent therapeutic effect for no recourse infections.

Accordingly, the invention results in a drug delivery system, formed as a tissue penetrating solution. The drug delivery system comprises: a solvent suitable for solubilizing a non-liquid active ingredient into a solution; a diluent for diluting the solvent to optimize the solution for mammalian tissue compatibility and a stabilizer for maintaining the solution chemically stable and substantially free from oxidation during storage for a pre-determined shelf life period. In some embodiments the solvent comprises a first tissue penetration enhancer, and may be the material known as dimethyl sulfoxide. The concentration ranges of dimethyl sulfoxide may include: a concentration range of between about 5% and 90%; a concentration range of between about 5% and 40%; a concentration range of between about 5% and 20%; a concentration range of between about 8% and 17%; a concentration range of between about 11% and 16%; or a concentration of about 15%. Preferably, the combined solvent within the drug delivery system has a diffusion constant greater than D=1.5×10⁻⁵ cm²/sec. In one embodiment of the preferred formulation, the diffusion constant was measured as approximately D=1.66×10⁻⁵ cm²/sec. This value agrees reasonably well with data measured by other researchers for basic DMSO in water, where the value of D was measured over a range of DMSO mole fractions, which range is incorporated herein as well by reference.

In the drug delivery system of the invention, the diluent may also have a characteristic of being a tissue penetration enhancer. Also, the diluent may have a diffusion constant that is different than the diffusion constant of the solvent. This is useful in forming a tissue penetrating drug delivery system compatible with various tissue types. In one embodiment of the drug delivery system, the diluent is dipropylene glycol. Various ratios of solvent to diluent are foreseen depending on the embodiment that is needed. In some embodiments, the ratio of solvent to diluent is between 1:5 and 1:1; while in other embodiments the ratio of solvent to diluent may be between 3:5 and 4:5. In other embodiments, particularly when a gel or other thickening agents are added, then the solvent to diluent ratio is altered to between about 5:1 and 20:1.

The drug delivery system stabilizer is selected from the list of stabilizers comprising ascorbic acid, sorbic acid, vitamin D and numerous other medically acceptable substitutes, including is selected from the list of dispersants including ascorbic acid, sorbic acid, a thiol, lipoic acid, a polyphenol, glutathione, tocopherol (vitamin E), a tocotrienal, uric acid, a peroxidase, coenzyme Q, carotene, and melatonin.

Also, the drug delivery system may be claimed as comprising at least one active pharmaceutical ingredient in the solution. In this instance, various embodiments are foreseen. For example, in some embodiments the active pharmaceutical ingredient may be selected from the list comprising anti-microbials, anti-virals, anti-fungals, anti-venoms. In further embodiments, the at least one active pharmaceutical ingredient comprises an anti-microbial ingredient selected from the list comprising tetracycline, doxycycline, or minocycline. Natural anti-microbial and anti-fungal ingredients, including, for example, thyme and other herbs and natural substances can be included in related embodiments. Even further embodiments comprise the at least one active pharmaceutical ingredient being tetracycline in a concentration of less than or equal to 3 percent.

The drug delivery system may be further enhanced by a controllable dispersion of the active agent throughout the solution by means of a dispersion enhancer. In one embodiment this may be achieved by configuring the stabilizer to function as a dispersion enhancer for dispersing the active agent in the solution.

In certain medical applications it is desirable to configure the drug delivery system as an ointment or similar semi-solid physical form. In such instances, the drug delivery system of the invention further comprises a semi-solid gel carrier formulated for solution mixing with the active ingredient, the solvent, the diluent, and the stabilizer; and with the gel carrier comprising oil-based gel. Alternatively, the drug delivery system of the invention may comprise a semi-solid gel carrier formulated for solution mixing with the active ingredient, the solvent, the diluent, and the stabilizer; with the gel carrier comprising water-based gel. In this embodiment, the semi-solid gel carrier may comprise water, glycerin, hydroxyethylcellulose, chlorhexidine digluconate, glucolactone, methylparaben, and sodium hydroxide in suitable proportions to form a semi-solid ointment with the active ingredient, the solvent, the diluent, and the stabilizer.

Alternatively, the drug delivery system of the invention may comprise a semi-solid gel carrier formulated for solution mixing with the active ingredient, the solvent, the diluent, and the stabilizer; with the gel carrier comprising a commercially-available gel, such as that product sold under the trade name K-Y Jelly or other commercial products that mix well with our basic formulation and are widely used by the general public and in the medical profession. Disadvantages of such ointments may include the addition of water with attendant degradation acceleration for certain active agents, and reduced biocidal activity due to higher water activity levels such additives impart.

In another embodiment, the invention comprises a drug delivery system, formed as a tissue penetrating solution, comprising: a solvent suitable for solubilizing a non-liquid pharmaceutical ingredient into a solution, the solvent comprising a first tissue penetration enhancer; a diluent for diluting the solvent to optimize the solution for mammalian tissue compatibility, the diluent comprising a second tissue penetration enhancer; and a stabilizer for maintaining the solution chemically stable and substantially free from oxidation degradation during storage for a pre-determined shelf life period, the stabilizer comprising a dispersion enhancer for dispersing the pharmaceutical ingredient in the solution. In this embodiment, it is possible for the solvent to comprise dimethyl sulfoxide, the diluent to comprise dipropylene glycol, and the stabilizer to comprise ascorbic acid. If further desired to form this drug delivery system into an ointment, then it may further comprise a semi-solid gel carrier formulated for solution mixing with the active ingredient, the solvent, the diluent, and the stabilizer, the gel carrier selected from the list comprising oil based gels and water based gels as described hereinabove. This description can result in either a water soluble or a non-water soluble product. Both have specific application for treatment of specific skin disorders and infections.

Yet another embodiment of the invention comprises a tissue penetrating drug delivery system, formed as a solution, comprising: a tissue penetrating solvent suitable for solubilizing a non-liquid active pharmaceutical ingredient, the solvent comprising dimethyl sulfoxide in a concentration range of between about 5% and 20%; a tissue penetrating diluent for diluting the solvent to optimize the solution for mammalian tissue compatibility, the diluent comprising dipropylene glycol (DPG) in a concentration range of between about 95% and 80%; and a stabilizer for maintaining the solution chemically intact and substantially free from oxidation during a pre-determined shelf life period, the stabilizer comprising ascorbic acid in a concentration range of between about 0.1% and 2%.

A still further embodiment of the invention includes an antibiotic medication for mammalian use, the antibiotic medication comprising a tissue penetrating drug delivery system formed in a solution with a 3% concentration tetracycline active pharmaceutical ingredient; the drug delivery system comprising a tissue penetrating solvent suitable for solubilizing a non-liquid active pharmaceutical ingredient, the solvent comprising dimethyl sulfoxide in a concentration range of between about 5% and 20%; a tissue penetrating diluent for diluting the solvent to optimize the solution for mammalian tissue compatibility, the diluent comprising dipropylene glycol in a concentration range of between about 95% and 80%; and a stabilizer for maintaining the solution chemically intact and substantially free from oxidation during a pre-determined shelf life period, the stabilizer comprising ascorbic acid in a concentration range of between about 0.1% and 2%. Similar to other embodiments, there may further be included an oil-based or water-based gel to provide different physical characteristics. It is recognized that such different physical characteristics may also impart altered drug delivery characteristics in view of the potentially larger volume or depot which a semi-solid form may create adjacent to an application site of tissue.

Yet another embodiment of the invention includes a medical aid kit for treating a penetrating wound injury. The wound injury may vary from a snake or other bite all the way to an injury to the skin barrier formed by a surgical placement of a medical device component, such as a pin element of an external fixation device. The medical aid kit may comprise; a first dispenser comprising a medical grade surfactant and disinfectant solution for applying to a contaminated surface having tissue toxic pathogens so that the pathogens are rendered substantially non-toxic and are removed from the contaminated surface; and a second dispenser comprising a medical grade antibiotic medication for applying to the contaminated surfaces comprising a tissue penetrating drug delivery system formed in a solution with a 3% concentration tetracycline active pharmaceutical ingredient; the drug delivery system comprising a tissue penetrating solvent suitable for solubilizing a non-liquid active pharmaceutical ingredient, the solvent comprising dimethyl sulfoxide in a concentration range of between about 5% and 20%; a tissue penetrating diluent for diluting the solvent to optimize the solution for mammalian tissue compatibility, the diluent comprising dipropylene glycol in a concentration range of between about 95% and 80%; and a stabilizer for maintaining the solution chemically intact and substantially free from oxidation during a pre-determined shelf life period, the stabilizer comprising ascorbic acid in a concentration range of between about 0.1% and 3% In use, the antibiotic medication protects the wound injury from re-infection due to pre-existing or subsequent introduction of pathogens. When the kit includes the antibiotic medication in the form of a semi-solid gel, as described herein, it is possible to form a barrier around or adjacent to the injured skin, such as around the circumference of a medical device fixator pin at the location of the pin penetration through the skin of a patient. This prevents migration of pathogens into the wound site by a mechanical barrier method while also driving the penetration of the active pharmaceutical agent (i.e. antimicrobial agent) down along the path of the device beneath the surface of the skin.

Yet another embodiment of the invention comprises a controllable volume penetration drug delivery system, formed as a solution, and suitable for delivering at least one active pharmaceutical ingredient to desired volumes of mammalian tissue adjacent to the site of application of the drug delivery system, comprising: a solvent suitable for solubilizing an active pharmaceutical ingredient, the solvent comprising a first diffusion constant suitable for carrying the solubilized active pharmaceutical throughout a first tissue volume within mammalian tissue; and a diluent for diluting the solvent and optimizing the solution for mammalian tissue compatibility, the diluent comprising a second diffusion constant suitable for carrying said active pharmaceutical ingredient throughout a second tissue volume within mammalian tissue. The drug delivery system may include further a stabilizer for maintaining the solution chemically stable and substantially free from degradation during a pre-determined shelf life period.

Tissue Healing, Regeneration and Sun Protection Ingredients

Additional tissue regeneration and repair ingredients may be added, comprising levels of ascorbic acid up to about 10 percent and medically efficacious amounts of Vitamin D, and preferably variants related to Vitamin D3. As demonstrated in zone of inhibition analysis against the various pathogens described herein, the drug delivery system demonstrates large multiples of kill zone measured area as compared with the area of the actual application of the drug delivery system. The resulting ratio of the areas of the zones of inhibition versus the areas of the zones of application are significant and not elsewhere shown. Accordingly, in one embodiment in which the solvent and diluent have first and second diffusion constants respectively, having individual benefits and a combined benefit of an optimum combined diffusion constant, and the ratio of an area of diffusion of the solution containing the active pharmaceutical agent as compared with the area of application of the solution is greater than 400%.

Embodiments of the inventions further comprise an ingredient to promote a third therapeutic effect of tissue healing using ingredients selected from the list of ingredients including Vitamin D, cholecalciferol, 7-dehydrocholesterol, 25-hydroxycholecalciferol, and 1,25-dihydroxycholecalciferol in a therapeutically efficacious amount. Additional possible ingredients may further comprise at least one homeopathic non-USP pharmaceutical-regulated ingredient to promote a third therapeutic effect of tissue healing selected from the list of ingredients including calcarea sulfurica, silica, D-glucuronicacid, vitamin A, vitamin E, vitamin C, bioflavonoids, garlic, garlic extract, coconut oil, tea-tree oil, oregano, colloidal silver, Arnica montana, aspirin, thymol, a mixture of cavacrol and thymol, oil of thyme, oil of lavender, Echinacea, marigold, myrrh, Symphytum officinale L., aloe vera, bromelain, and goldenseal in a therapeutically efficacious amount.

The invention has controllable features that enable remarkable drug delivery tunable characteristics. In one embodiment, a dual carrier controllable depth penetration drug delivery system is provided as a solution, and is suitable for delivering efficacious dosages of at least one active pharmaceutical ingredient to desired depths of mammalian tissue. The drug delivery system comprises: a first carrier suitable for solubilizing and carrying an active pharmaceutical ingredient through tissue, the first liquid carrier comprising a first diffusion constant suitable for carrying an efficacious concentration of an active pharmaceutical to a tissue depth deeper than the stratum corneum within a mammalian tissue site; and a second carrier suitable for both diluting the solvent and optimizing the solution for mammalian tissue compatibility, the second liquid carrier having a second diffusion constant different than the first diffusion constant and suitable for carrying an efficacious concentration of said active pharmaceutical ingredient to a tissue depth shallower than the stratum corneum within the mammalian tissue site. In this dual carrier controllable depth penetration drug delivery system, the first carrier may have a diffusion constant greater than about 1.5×10⁻⁵ cm²/sec and the second carrier may have a lesser diffusion constant. The first carrier may be dimethyl sulfoxide, while the second carrier may be dipropylene glycol. The dual carrier controllable depth penetration drug delivery system may further comprise a dispersion agent for controlling the dispersion and concentration of the active pharmaceutical ingredient at different depths of tissue penetration of the drug delivery system.

In a further embodiment, it is possible to add one or more sunscreen or sunblock agents to the formulation of the invention. A key factor enabling this embodiment is the compatibility of these agents with dipropylene glycol and with the dimethyl sulfoxide. For example, the following sunscreen or sunblock agents are conducive to use with this formulation, as desired, although additional such agents are contemplated within the scope of this invention: Aminobenzoic acid (PABA), Avobenzone, Cinoxate, Dioxybenzone, Homosalate, Menthyl anthranilate, Octocrylene, Octyl methoxycinnamate, Octyl salicylate, Oxybenzone. Padimate, Phenylbenzimidazole sulfonic acid, Sulisobenzone, Titanium dioxide, Trolamine salicylate, and Zinc oxide.

The ability of the novel formulation of the invention to provide excellent tissue penetration enables use of the above agents in novel and unforeseen ways. For example, it may be possible to utilize less than a normal preferred dosage of one or more of these agents while achieving excellent protective effects. In one embodiment, the preferred dosage of each of the above agents is: Aminobenzoic acid (PABA) up to 15 percent, Avobenzone up to 3 percent, Cinoxate up to 3 percent, Dioxybenzone up to 3 percent, Homosalate up to 15 percent, Menthyl anthranilate up to 5 percent, Octocrylene, Octyl methoxycinnamate, Octyl salicylate up to 5 percent, Oxybenzone up to 6 percent, Padimate up to 8 percent, Phenylbenzimidazole sulfonic acid up to 4 percent, Sulisobenzone up to 10 percent, Titanium dioxide up to 25 percent, Trolamine salicylate up to 12 percent, Zinc oxide up to 25 percent.

A further embodiment of the invention is to use both a sunscreen or sunblock agent as well as the additional Vitamin D source, referred to herein. In one embodiment, the invention includes a controllable volume penetration drug delivery system. This is formed as a solution, and is suitable for delivering at least one active pharmaceutical ingredient to desired volumes of mammalian tissue adjacent to the site of application of the drug delivery system. The system further includes a tissue regeneration system for improving the health of tissue adjacent to the site of application of the drug delivery system, comprising. The tissue protection and regeneration system comprises a solvent suitable for solubilizing an active pharmaceutical ingredient and one or more tissue protection and regeneration ingredients. The solvent comprises a first diffusion constant suitable for carrying the solubilized active pharmaceutical and other ingredients throughout a first tissue volume within mammalian tissue. A diluent is provided for diluting the solvent and optimizing the solution for mammalian tissue compatibility, with the diluent comprising a second diffusion constant suitable for carrying said active pharmaceutical and other ingredients throughout a second tissue volume within mammalian tissue. The tissue protection and regeneration system comprises a sunscreen or sunblock agent as well as an oxygen stabilizer. The stabilizer is provided in a total concentration range of between about 3% and 10%, and a vitamin D source is added in a medically efficacious amount. Examples of a suitable vitamin D source include cholecalciferol, 7-dehydrocholesterol, 25-hydroxycholecalciferol, and 1,25-dihydroxycholecalciferol or an equivalent substance.

The extraordinary synergy of the above embodiment of the invention is appreciated in that a user of tetracycline or similar antibiotics may experience sensitivity to sunlight. If that occurs, then the addition of a sunscreen or sunblock agent may be useful. However, it is known that some users of sunscreen and sunblock agents may experience less generation of Vitamin D due to less ultraviolet penetration into the user's skin tissue. The novel combination of providing a Vitamin D source, a sunscreen or sunblock agent, and a powerful antibiotic with the drug delivery system of this invention, all in one formulation, overcomes the combination of prior problems with great efficacy. Moreover, when using the embodiment that does not include gelling or other semi-solid agents, the formulation does not leave the user with a feeling of having a residue or oily sensation on their skin. This embodiment may be preferred for use on the facial, neck and other areas of the user. This desired effect occurs because the penetrating agents draw the medication into the tissue and allow the user a more clean sensation on their outer skin layer. That is particularly important in facial or other normally exposed skin areas, and is particularly important to users with acne, rosacea or other skin maladies.

What is further provided is a method of formulating a shelf life stable solution at room temperature with wide process latitude using inherently unstable pharmaceutical active ingredients. In this method, the active ingredient is diluted with a solvent in proper ratios and then a stabilizer is added, while maintaining the solution. A diluent is then added, with additional materials then combined according to the desired viscosity and other characteristics.

1.-98. (canceled)

99. An over-the-counter (OTC) regulated therapeutic composition for self-medication use for application to wounds having a potential pathogen load, said composition comprising:
a tissue penetration enhancer;
a pharmaceutical agent; and
a hygroscopic carrier agent;
wherein the activity/water (A_{w}) measurement of the composition is less than the A_{w} measurement for a target pathogen in a tissue wound.

100. The composition of claim 99, wherein the penetration enhancer comprises a Class I pharmaceutical incipient.

101. The composition of claim 99, wherein the pharmaceutical agent is selected from the group consisting of antimicrobials, antifungals, antivirals, anesthesics, analgesics, corticosteroids, non-steroidal anti-inflammatories, retinoids, lubricating agents, anti-warts, anti-proliferative, vasoactive, keratolytic, dicarboxylic acids and esters; calcium channel blockers, cholinergic, N-oxide donors, photodynamic, anti-acne, anti-wrinkle, anti-oxidants, self-tanning active herbal extracts, acaricides, age spot and keratose removing agents, allergens, anti-aging agents, antibiotics, anti-burn agents, anti-cancer agents, anti-dandruff agents, anti-depressants, anti-dermatitis agents, anti-edemics, antihistamines, antihelminths, anti-hyperkeratolyte agents, anti-inflammatory agents, anti-irritants, anti-lipemics, antimycotics, anti-proliferative agents, anti-anti-pruritics, anti-psoriatic agents, anti-rosacea agents, anti-seborrheic agents, antiseptics, anti-swelling agents, anti-yeast agents, astringents, topical cardiovascular agents, chemotherapeutic agents, dicarboxylic acids, disinfectants, fungicides, hair growth regulators, hormones, hydroxy acids, immuno-suppressants, immuno-regulating agents, insecticides, insect repellents, keratolytic agents, lactams, metals, metal oxides, mitocides, neuropeptides, oxidizing agents, pediculicides, photodynamic therapy agents, retinoids, sanatives, scabicides, self-tanning agents, skin whitening agents, vasoconstrictors, vasodilators, vitamins, vitamin D derivatives, wound healing agents and wart removers.

102. The composition of claim 101, wherein the antibiotic is selected from the group consisting of tetracycline, doxycycline, and minocycline.

103. The composition of claim 99, wherein the penetration enhancer is non-hygroscopic.

104. The composition of claim 99, wherein the pharmaceutical agent is in a dosing suitable for use in an OTC listing.

105. The composition of claim 99, wherein the hygroscopic carrier comprises a Class I pharmaceutical incipient suitable for mixing in solution with the tissue penetration enhancer and the antibiotic agent.

106. The composition of claim 99, wherein the tissue penetration enhancer is a solvent

107. The composition of claim 106, wherein the solvent is selected from the group consisting of dimethyl sulfoxide and dodecyl methyl sulfoxide.

108. The composition of claim 99, wherein the hygroscopic carrier agent is a polyol.

109. The composition of claim 107, wherein the polyol is selected from the group consisting of propylene glycol, dipropylene glycol, polypropylene glycol, 1,2-propanediol, and polyethylene glycol.

110. The composition of claim 99, further comprising an antioxidant.

111. The composition of claim 109, wherein the antioxidant is selected from the group consisting of ascorbic acid, sorbic acid, a thiol, lipoic acid, a polyphenol, glutathione, tocopherol (vitamin E), a tocotrienal, uric acid, a peroxidase, coenzyme Q, carotene, and melatonin.

112. The composition of claim 99, wherein at least one of the penetration enhancer or hygroscopic carrier agent disrupts normal methylation and replication of target pathogens sufficient to provide a secondary effect to enable greater efficacy of the pharmaceutical antibiotic agent.

113. The composition of claim 99, wherein the penetration enhancer and the hygroscopic carrier agent have a first and second diffusion constant, respectively, that create a combined diffusion effect so that the surface area ratio of an area of diffusion of the solution is at least about 200 percent of the tissue surface area of application of the solution to the mammalian tissue.

114. The composition of claim 99, wherein the tissue penetration enhancer and the hygroscopic carrier agent are selected from sulfoxides, polyols, urea, sugars, lactams, amides, fatty acids, fatty alcohols, terpenes, anionic-surfactants, cationic-surfactants, non-ionic surfactants, and Zwitterionic-surfactants.

115. The composition of claim 99, wherein the composition is for mammalian use.

116. The composition of claim 99, wherein the composition comprises a tissue penetrating drug delivery system formed in a solution with a 3% concentration tetracycline active pharmaceutical ingredient and a tissue restoration system; said drug delivery system comprising:
a. a tissue penetrating solvent suitable for solubilizing a non-liquid active pharmaceutical ingredient, said solvent comprising dimethyl sulfoxide in a concentration range of between about 5% and 20%;
b. a tissue penetrating diluent for diluting the solvent to optimize the solution for mammalian tissue compatibility, said diluent comprising dipropylene glycol in a concentration range of between about 95% and 80%; and
c. a stabilizer for maintaining the solution chemically intact and substantially free from oxidation during a pre-determined shelf life period, said stabilizer comprising ascorbic acid in a concentration range of between about 0.1% and 3% inclusive;
and said tissue restoration system comprising enhanced stabilizer volume to increase total stabilizer concentration to 3% to 10% inclusive.

117. The composition of claim 99, further comprising a vitamin D.

118. A topical medicament comprising the composition of claim 99.

119. The topical medicament of claim 118, wherein the topical medicament is non-polymeric.

120. The medicament of claim 118, further comprising an ingredient to promote tissue healing.

121. The medicament of claim 120, wherein the ingredient is a homeopathic non-USP pharmaceutical-regulated ingredient.

122. A surgical medicament for use as a penetrating medicated lavage in a deep tissue wound, said medicament comprising a composition of claim 99.

123. A medical aid kit for treating a penetrating wound injury comprising a composition of claim 99.
